(19) United States
(12) Patent Application Publication
(10) Pub. No.: US 2022/0080122 A1
(43) Pub. Date: Mar. 17, 2022

(54) DEVICES AND METHODS FOR ADMINISTERING A THERAPEUTIC PREPARATION

(71) Applicant: INCUBE LABS, LLC, San Jose, CA (US)

(72) Inventors: Mir A. IMRAN, Los Altos Hills, CA (US); Arthur Hsu Chen CHANG, Irvine, CA (US)

(73) Assignee: INCUBE LABS, LLC, San Jose, CA (US)

(21) Appl. No.: 17/536,342

(22) Filed: Nov. 29, 2021

Related U.S. Application Data
(63) Continuation of application No. PCT/US2020/034883, filed on May 28, 2020.
(60) Provisional application No. 62/854,101, filed on May 29, 2019.

(51) Int. Cl. A61M 5/172; A61M 31/00

(52) U.S. Cl. CPC A61M 5/172; A61M 31/00; A61M 2205/3344; A61M 2205/3368; A61M 2205/3324; A61M 2205/3507; A61M 2210/1053; A61M 2210/106; A61M 2210/1064; A61M 2210/1017; A61M 2205/3327

(57) ABSTRACT

Systems, devices and methods are provided for administering a carrier incorporating a therapeutic preparation within a subject. A device includes a carrier, and a launch assembly to deploy the carrier by ejecting the carrier from the device and into internal tissue of the subject. The device detects a status of deployment of the carrier. A system includes a delivery device, a carrier disposed in the delivery device, a launch assembly, and detection circuitry. The launch assembly causes the carrier to exit the delivery device so as to penetrate internal tissue of the subject. The system determines a status of deployment of the carrier using the detection circuitry. A method includes introducing a launch assembly within the subject, the launch assembly coupled to a carrier; deploying, by the launch assembly, the carrier; and detecting a status of deployment of the carrier.

### CROSS-REFERENCE TO RELATED APPLICATIONS

This application is a continuation of U.S. National Phase of PCT Application No. PCT/US2020/034883 filed on May 28, 2020 which, in turn, claims the benefit of U.S. Provisional Application No. 62/854,101 filed on May 29, 2019, the disclosures of which are hereby incorporated in their entirety by reference herein.

### BACKGROUND

Therapeutic preparations can be delivered to a subject in various ways such as by way of enteral or parenteral delivery. For many delivery techniques, it can be helpful to be able to monitor characteristics of the delivery and the delivery environment.

### SUMMARY

Systems, devices and methods are provided for administering a therapeutic preparation within a subject. In an embodiment, a device includes a carrier including a therapeutic preparation, and a launch assembly to deploy the carrier by ejecting the carrier from the device and into internal tissue of the subject. The device detects a status of deployment of the carrier. In an embodiment, a system includes a delivery device, a carrier disposed in the delivery device, a launch assembly, and detection circuitry. The carrier includes a therapeutic preparation. The launch assembly causes the carrier to exit the delivery device so as to penetrate internal tissue of the subject. The system determines a status of deployment of the carrier using the detection circuitry. In an embodiment, a method includes introducing a launch assembly within the subject, the launch assembly coupled to a carrier that incorporates the therapeutic preparation; deploying, by the launch assembly, the carrier; and detecting a status of deployment of the carrier. Further details of these and other embodiments and aspects of the invention are described more fully below, with reference to the attached drawing figures.

### BRIEF DESCRIPTION OF THE DRAWINGS

The accompanying drawings are not intended to be drawn to scale. Like reference numbers and designations in the various drawings indicate like components. For purposes of clarity, not every component may be labeled in every drawing. In the drawings:

FIG. 1 is a block diagram of a system for administering a therapeutic preparation within a subject, in an embodiment.

FIG. 2A illustrates an example chamber including an example detector, in an embodiment.

FIG. 2B illustrates an example chamber including an example detector, in an embodiment

FIG. 2C illustrates an example detector that can be used to implement the detector discussed above in relation to FIG. 2B, in an embodiment

FIG. 3A illustrates an example chamber including an example detector, in an embodiment.

FIG. 3B illustrates the example chamber of FIG. 3A during deployment.

FIG. 4A illustrates an example chamber including an example detector, in an embodiment.

FIG. 4B illustrates the example chamber of FIG. 4A during deployment.

FIG. 4C illustrates a frame with extensor for triggering a detection of deployment.

FIG. 5 illustrates an example detector, in an embodiment.

FIG. 6 illustrates an example chamber including an example detector, in an embodiment.

FIG. 7 illustrates an example carrier, in an embodiment.

FIG. 8 illustrate an example carrier, in an embodiment.

FIG. 9 illustrates a block diagram of the first communication device discussed above in relation to FIG. 1, in an embodiment.

FIG. 10 illustrates an example belt that can be used with a first communication device shown in FIGS. 1 and 9, in an embodiment.

FIG. 11 is a flow diagram of a first example process, in an embodiment.

FIG. 12 is a flow diagram of a second example process, in an embodiment.

FIG. 13 is a flow diagram of a third example process, in an embodiment.

### DETAILED DESCRIPTION

When used in the present disclosure, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the singular terms “a,” “an,” and “the” may include plural referents unless the context clearly dictates otherwise. Reference to an object in the singular is not intended to mean “one and only one” unless explicitly so stated, but rather “one or more.”

References to “or” may be construed as inclusive so that any terms described using “or” may indicate any of a single, more than one, and all of the described terms.

The term “in an embodiment” or a variation thereof (e.g., “in another embodiment” or “in one embodiment”) refers herein to use in one or more embodiments, and in no case limits the scope of the present disclosure to only the embodiment as illustrated and/or described. Accordingly, a component illustrated and/or described herein with respect to an embodiment can be used in another embodiment (e.g., in another embodiment illustrated and described herein, or in another embodiment within the scope of the present disclosure and not illustrated and/or not described herein).

The term “component” refers herein to one item of a set of one or more items that together make up a device, formulation, or system under discussion. A component may be in a solid, powder, gel, plasma, fluid, gas, or other form. For example, a device may include multiple solid components which are assembled together to structure the device and may further include a liquid component that is disposed in the device. For another example, a formulation may include two or more powdered and/or fluid components which are mixed together to make the formulation.

The term “design” or a grammatical variation thereof (e.g., “designing” or “designed”) refers herein to characteristics intentionally incorporated into a design based on, for example, estimates of tolerances related to the design (e.g., component tolerances and/or manufacturing tolerances) and estimates of environmental conditions expected to be encountered by the design (e.g., temperature, humidity, external or internal ambient pressure, external or internal mechanical pressure, external or internal mechanical stress, age of product, physiology, body chemistry, biological composition of fluids or tissue, chemical composition of fluids or tissue, pH, species, diet, health, gender, age, ancestry, disease, tissue damage, shelf life, or the combination of such); it is to be understood that actual tolerances and environmental conditions before and/or after delivery can affect such designed characteristics so that different components, devices, formulations, or systems with a same design can have different actual values with respect to those designed characteristics. Design encompasses also variations or modifications to the design, and design modifications implemented after manufacture.

The term “manufacture” or a grammatical variation thereof (e.g., “manufacturing” or “manufactured”) as related to a component, device, formulation, or system refers herein to making or assembling the component, device, formulation, or system. Manufacture may be wholly or in part by hand and/or wholly or in part in an automated fashion.

The term “structured” or a grammatical variation thereof (e.g., “structure” or “structuring”) refers herein to a component, device, formulation, or system that is manufactured according to a concept or design or variations thereof or modifications thereto (whether such variations or modifications occur before, during, or after manufacture) whether or not such concept or design is captured in a writing.

The term “body” refers herein to an animalia body.

The term “subject” refers herein to a body into which a delivery device is, or is intended to be, delivered. For example, with respect to humans, a subject may be a patient under the treatment of a health care professional.

The term “biological matter” refers herein to blood, tissue, fluid, enzymes, and other secretions of a body. The term “digestive matter” refers herein to biological matter along the GI tract in an animalia body, and other matter (e.g., food in an undigested or a digested form such as chyme) traversing the gastrointestinal tract.

The term “therapeutic preparation” refers herein to a medicinal preparation (e.g., including one component or a combination of components) intended for a therapeutic, diagnostic, or other biological purpose in any form. A therapeutic preparation may be in a liquid form, a powder form, or a condensed or a consolidated form such as a tablet or microtablet. Each therapeutic preparation can include one or more components, and a device or system can include one or more therapeutic preparations. A component of a therapeutic preparation can be, for example, a pharmacologically active agent, a deoxyribonucleic acid (DNA) or small interfering ribonucleic acid (SiRNA) transcript, a cell, a cytotoxic agent, a vaccine or other prophylactic agent, a nutraceutical agent, a vasodilator, a vasoconstrictor, a delivery enhancing agent, a delay agent, an excipient, a diagnostic agent, or a substance for cosmetic enhancement.

A pharmacologically active agent can be, for example, an antibiotic, a nonsteroidal anti-inflammatory drug (NSAID), an angiogenesis inhibitor, a neuroprotective agent, a chemotherapeutic agent, a peptide, a protein, an immunoglobulin (e.g., a TNF-alpha antibody), an interleukin in the IL-17 family of interleukins, an anti-eosinophil antibody, another antibody, a large molecule, a small molecule, or a hormone, or a biologically active variant or derivative of any of the foregoing.

A cell can be, for example, a stem cell, a red blood cell, a white blood cell, a neuron, or other viable cell. Cells can be produced by or from living organisms or contain components of living organisms. A cell can be allogeneic or autologous.

A vaccine can be, for example, against an influenza, a coronavirus, meningitis, human papillomavirus (HPV), or chicken pox. A vaccine can correspond to an attenuated virus.

A nutraceutical agent can be, for example, vitamin A, thiamin, niacin, riboflavin, vitamin B-6, vitamin B-12, another B-vitamin, vitamin C (ascorbic acid), vitamin D, vitamin E, folic acid, phosphorous, iron, calcium, or magnesium.

A vasodilator can be, for example, 1-arginine, sildenafil, a nitrate (e.g., nitroglycerin), or epinephrine.

A vasoconstrictor can be, for example, a stimulant, an amphetamine, an antihistamine, epinephrine, or cocaine.

A delivery enhancement agent can be, for example, a permeation enhancer, an enzyme blocker, a peptide that permeates through mucosa, an antiviral drug such as a protease inhibitor, a disintegrant, a superdisintegrant, a pH modifier, a surfactant, a bile salt, a fatty acid, a chelating agent, or a chitosan. A delivery enhancing agent can, for example, serve as a delivery medium for delivery of a component of a therapeutic preparation, or serve to improve absorption of a component of a therapeutic preparation into the body. A delivery enhancing agent can prime an epithelium of the intestine (e.g., fluidize an outer layer of cells) to improve absorption and/or bioavailability of one or more other components included in the delivery device.

A delay agent can be, for example, poly(lactic acid) (PLA), poly(glycolic acid) (PGA), polyethylene glycol (PEG), poly(ethylene oxide) (PEO), poly (l-lactic acid) (PLLA), poly(D-lactic acid) (PDLA), another polymer, or a hydrogel. A delay agent can be included with (e.g., mixed with, or providing a structure around) one or more other component(s) in a therapeutic preparation to slow a release rate of the other component(s) from the therapeutic preparation.

An excipient can be, for example, a binder, a disintegrant, a superdisintegrant, a buffering agent, an anti-oxidant, or a preservative. Excipients can provide a medium for a component of a therapeutic preparation (e.g., for assisting in manufacture), or to preserve integrity of a component of a therapeutic preparation (e.g., during manufacture, during storage, or after ingestion prior to dispersion within the body).

A diagnostic agent can be, for example, a sensing agent, a contrast agent, a radionuclide, a fluorescent substance, a luminescent substance, a radiopaque-substance, or a magnetic substance.

The term “ingest” or a grammatical variation thereof (e.g., “ingesting” or “ingested”) refers herein to taking into the stomach, whether by swallowing or by other means of depositing into the stomach (e.g., by depositing into the stomach by endoscope or depositing into the stomach via a port).

The term “fluid” refers herein to a liquid, and encompasses moisture and humidity. The term “fluidic environment” refers herein to an environment in which one or more fluids are present.

The terms “substantially” and “about” are used herein to describe and account for small variations. For example, when used in conjunction with a numerical value, the terms can refer to a variation in the value of less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

As used herein, a range of numbers includes any number within the range, or any sub-range if the minimum and maximum numbers in the sub-range fall within the range. Thus, for example, “<9” can refer to any number less than nine, or any sub-range of numbers where the minimum of the sub-range is greater than or equal to zero and the maximum of the sub-range is less than nine. Ratios may also be presented herein in a range format. For example, a ratio in the range of about 1 to about 200 should be understood to include the explicitly recited limits of about 1 and about 200, and also to include individual ratios such as about 2, about 35, and about 74, and sub-ranges such as about 10 to about 50, about 20 to about 100, and so forth.

Therapeutic preparations can be delivered to a subject in various ways such as by way of oral or enteral delivery, or such as by way of parenteral delivery (e.g., intravenous, rectal, subcutaneous, transdermal, or via implant). For many techniques of delivery, it can be useful to monitor aspects of the delivery, such as environmental conditions before, during, and/or after the delivery, time of delivery, information about the therapeutic preparation delivered, and/or delivery location.

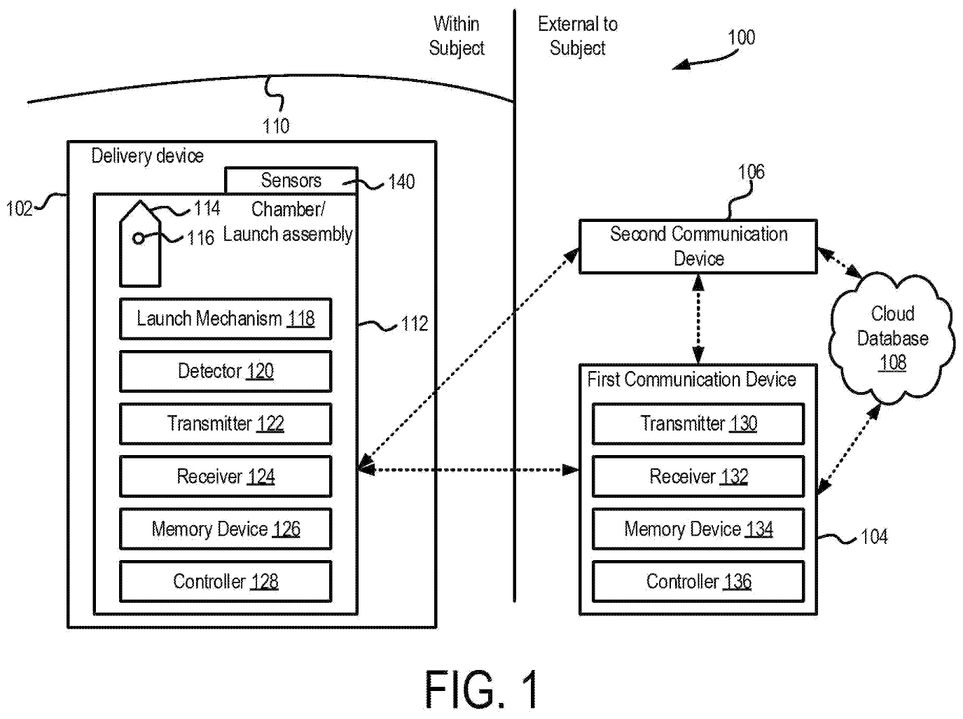

FIG. 1 illustrates a block diagram of an embodiment of a system 100 for administering a therapeutic preparation within a subject (e.g., a patient undergoing surgery, treatment, or therapy), and monitoring aspects of the administration of the therapeutic formulation and/or aspects of an environment. The system 100 includes a delivery device 102 and may include a first communication device 104 and/or a second communication device 106.

The delivery device 102 can be structured to deliver a therapeutic preparation to a body in general without a specific target delivery site, or for delivering a therapeutic preparation to a specific location within the body. The delivery device 102 can incorporate any container or structure that can be inserted or otherwise introduced into the body. In an embodiment, the delivery device 102 can be designed and structured to be delivered and/or positioned adjacent to a delivery site 110 within the body where the therapeutic preparation is intended to be delivered. The delivery site 110 could be at a subcutaneous site, an intramuscular site, a site within a cavity or organ, a site within a lumen, or other site within a body.

In an embodiment, the delivery site 110 is within a gastrointestinal (GI) tract of the subject. In an embodiment, the delivery site 110 is along a wall of the stomach, small intestine or large intestine. In an embodiment, delivery is into, or through, a wall of the GI tract; for example, delivery may be into or through a mucosal layer, into or through a sub-mucosal layer, into or through a muscular layer, into or through the serosa, into or through the peritoneum, into the peritoneal cavity, or into the mesentery or another organ in the peritoneal cavity.

In an embodiment, injection or surgical placement is used to position the delivery device 102 at or adjacent to the delivery site 110.

In an embodiment, the delivery device 102 includes, or is incorporated into, a capsule that can be orally ingested by the subject.

In an embodiment, the delivery device 102 includes a launch assembly 112 to deliver a payload to the delivery site 110. The assembly 112 may in general refer to a collection of one or more components of the delivery device 102. The assembly may include a container (e.g., a chamber) in which one or more components of the delivery device 102 are disposed.

The assembly 112 can house a carrier 114 structured to deliver a payload 116 within a subject's body. The payload 116 can include a therapeutic preparation and/or electronics.

The carrier 114 can have a number of shapes and forms, including needle-shaped, dart-like, cylindrically-shaped, cone-like, diamond-shaped, pyramidal-shaped, box-shaped, or other shape. The carrier 114 can be solid, or can define a hollow portion within the carrier 114. In an embodiment, the carrier 114 is structured to penetrate tissue and thus includes an end having a tissue penetrating shape, for example a tapered or pointed end; in another embodiment, the carrier 114 is not structured to penetrate tissue. In an embodiment, the carrier 114 is structured to be adherent to tissue (e.g., has adhesive qualities due to an adhesive substance being applied, or has adhesive qualities due to surface characteristics such as roughness). In an embodiment, the carrier 114 contains the payload 116; in another embodiment, the carrier 114 is itself the payload 116, meaning that the therapeutic formulation of the payload 116 is formed into a shape that is the carrier 114.

In the embodiment illustrated in FIG. 1, the assembly 112 includes a launch mechanism 118, a detector 120, a transmitter 122, a receiver 124, a memory device 126, and/or a controller 128.

The launch mechanism 118 can be structured to deploy (e.g., move, exit, reveal, release, and/or launch) the carrier 114 from the delivery device 102. For example, the launch mechanism 118 may cause the carrier 114 to fully exit the delivery device 102, or may position the carrier 114 at least partially out of the delivery device 102, or at least partially out of the assembly 112, or may reveal the carrier 114 to biological matter (or digestive matter) present at the delivery site.

In an embodiment, the launch mechanism 118 is structured to launch the carrier 114 out of the delivery device 102 and into tissue of the GI tract such as a wall of the GI tract (e.g., into a layer of the wall, or through one or more layers of the wall, or through the wall and into or through the peritoneal membrane).

In an embodiment, the detector 120 corresponds to one or more detectors that are structured to detect a deployment of the carrier 114.

In an embodiment incorporating the transmitter 122, the transmitter 122 can transmit data to at least one communication device external to the subject. In an embodiment, the transmitter 122 includes a low-power oscillator circuit operating in the 10 megahertz (MHz) to 50 MHz range, more preferably in the 35 MHz to 45 MHz range, which may minimize radiofrequency (RF) wave absorption in the human body. In an embodiment, the transmitter 122 may include a tuned oscillator for amplitude modulation for improved specificity and thus improved detection of the signal.

In an embodiment incorporating the receiver 124, the receiver 124 can receive data from at least one communication device external to the subject (e.g., the first communication device 104 and/or the second communication device 106).

The memory device 126 can store data related to the delivery device, such as data received from the detector 120, one or more sensors 140 incorporated with the delivery device 102, the first communication device 104, and/or the second communication device 106. The memory device 126 can also store pre-loaded information, such as lot information of the therapeutic preparation (or payload 116), the assembly 112, the carrier 114, and/or the delivery device 102. Data in the memory device 126 may be subsequently retrieved, such as by a download of the data, or such as by the delivery device 102 transmitting the data by way of the transmitter 122.

The controller 128 can be programmed to control operation of various of the components of the assembly 112. The controller 128 may, for example, correspond to one or more of a microcontroller, an FPGA (field programmable gate array), an ASIC (application-specific integrated circuit), other integrated circuit, an analogue-based controller, or a combination of the foregoing. In an embodiment, the controller 128 includes the memory device 126. While not shown, the assembly 112 can include or otherwise be operatively coupled to a power source such as a battery. In particular embodiments, the battery may be or may include a lithium ion battery having a voltage of around 1.5 volts. At least one antenna (not shown) associated with the assembly 112 can be used to transmit signals to one or more devices external to the subject, and/or to receive signals from one or more devices external to the subject (e.g., the first communication device 104 and/or the second communication device 106).

In an embodiment, at least one antenna associated with the assembly 112 can be used to receive power from an external power source for powering one or more components associated with the system 100 (e.g., one or more of the launch mechanism 118, the detector 120, the transmitter 122, the receiver 124, the memory device 126, the controller 128, or the sensor(s) 140) and/or to recharge a battery operatively coupled to the assembly 112. In an embodiment, at least one antenna associated with the assembly 112 can both receive power and communicate signals.

In an embodiment, the assembly 112 can include an electronics module to incorporate, host, localize, interconnect and/or embed circuitry or electronics of at least one of the following: the launch mechanism 118, the detector 120, the transmitter 122, the receiver 124, the memory device 126, the controller 128, the sensor(s) 140, the power source, or the antenna(s). The electronics module can include one or more circuitry boards (e.g., printed circuited boards), integrated circuits, and/or discrete electronic components, and can couple to any portion of the assembly 112 or components thereof, or any portion of the delivery device 102.

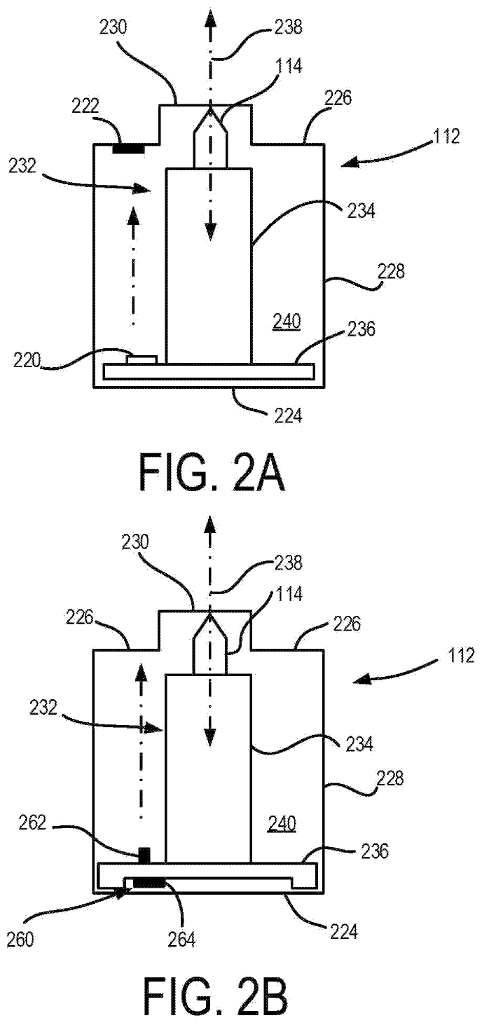

In an embodiment, the electronics module corresponds to a printed circuit board that is substantially in the shape of a disc or annular ring (e.g., with respect to FIG. 2A or FIG. 2B, conformed to a shape of the base 236, and can be mounted on the base 236 or can form part of the structure of the base 236).

In an embodiment, one or more of the sensors 140 can be coupled to the controller 128, such as to aid in timing, and/or monitoring, deployment of the carrier 114.

In an embodiment, the sensors 140 may be coupled to the assembly 112 or elsewhere on or within the delivery device 102. The sensors 140 can include, for example, one or more of, or a combination of, a pressure sensor, velocity sensor, accelerometer, orientation sensor, pH level sensor, torsion sensor for sensing torsion on walls of the delivery device 102, sensor for sensing breach or penetration or tension of a penetrable portion of the delivery device 102 (e.g., a breach surface such as the breach surface 230 in FIG. 2A and FIG. 2B)), vibration sensor, moisture sensor, fluid conductivity sensor, fluid chemistry sensor, fluid composition sensor, or location sensor.

In an embodiment, the controller 128 can be communicably coupled to the detector 120 and/or the sensors 140 to receive data or signals. The controller 128 can store the received data or signals in the memory device 126, and/or can cause the received date or signals to be transmitted by way of transmitter 122.

In an embodiment, the delivery device 102 can be an inflatable device that can be structured to unfurl and/or inflate when the delivery device 102 is in a desired position within the subject, such as in proximity to a delivery site 110 in a target region (e.g., a wall of the GI tract). A target region in the GI tract can be, for example, a region in an abdominal cavity, digestive system, large intestine region, cecum region, colon region, small intestine region, small bowel region, duodenum region, jejunum region, ileum region, ulcer region, cyst region, infection region, GI perforation region, intestinal lining region, or intestinal wall region. The inflatable device can inflate, align, and/or stabilize the assembly 112 in a desired position in relation to the delivery site 110 such that deployment of the carrier 114 from the assembly 112 will likely result in the carrier 114 making contact with the delivery site 110. In an embodiment, the delivery device 102 can include a launch mechanism 118 such as a compressed air source, an electromagnetic solenoid, a spring mechanism, or any other mechanism that can impart a propulsive force to eject the carrier 114 from the assembly 112 (e.g., a propulsive force against the base 236 in FIG. 2A-FIG. 2B).

In an embodiment, the delivery device 102 can further include a monitoring device structured to detect one or more predefined conditions, and to initiate deployment of the carrier 114 responsive to detecting the one or more predefined conditions. In an embodiment, a predefined condition is that a value of a parameter sensed by one of the sensors 140 is below or above a predefined threshold value. As an example, the delivery device 102 may sense a pH level of fluid outside the delivery device 102 to detect a current location of the delivery device 102. For instance, pH levels in the intestine can be greater than pH levels elsewhere in the GI tract preceding the intestine. When the delivery device 102 senses that pH levels have risen above a threshold value, the delivery device 102 can position, align, and/or stabilize the delivery device 102 or a portion thereof against a wall of the intestine (here, the delivery site 110), and can then activate the deployment mechanism to deploy the carrier 114 into the wall. The delivery device 102 can then detect the activation of the deployment mechanism and/or detect deployment of the carrier 114.

In an embodiment, the sensors 140 can include one or more sensors 140 to determine orientation of the delivery device 102 (e.g., a gyroscope, accelerometer, or microelectromechanical systems (MEMS) accelerometer). In use, an embodiment of the delivery device 102 can have at least one orientation sensor 140 that allows the delivery device 102 and/or the system 100 to detect motion of the delivery device 102 during traversal of the GI tract of the subject based on input from the orientation sensor; for example, by tracking motion, location, and/or orientation of the delivery device 102 within the GI tract over time, the sensors 140 can detect if the delivery device 102 has reached or is near a target region. The delivery device 102 can also monitor the orientation (e.g., up, down, or sideways) of the delivery device 102 in relation to the subject or some other frame of reference. As an example, in embodiments in which the delivery device 102 is an inflatable balloon and the carrier 114 is ejected from the assembly 112 into a wall of the GI tract, the delivery device 102 can ensure that upon inflation, an orientation of an ejection path of the assembly 112 is approximately perpendicular to the wall.

In an embodiment, the sensors 140 include one or more sensors (e.g., a proximity, pressure, and/or orientation sensor) to determine a position of the carrier 114 relative to that of the assembly 112, such as to detect if the carrier 114 has been deployed from the assembly 112, or if it is still held in place within the assembly 112.

In an embodiment, the sensors 140 can include one or more moisture sensors. For example, the delivery device 102 can be a capsule (e.g., a tablet, cylindrical, or low-drag form of capsule) that can be orally ingested by the subject. When the capsule reaches a target region, the capsule dissolves, causing fluid in the GI tract to make contact with other components of the delivery device 102. The delivery device 102 can detect the presence of moisture (e.g., due to the fluid in the GI tract), and can initiate deployment of the carrier 114. In an embodiment, an outer surface of the delivery device 102 can include one or more contacts that are coated with an enteric coating. The exposure of the delivery device 102 to the fluids in the GI tract can cause the enteric coating to dissolve, exposing the contacts. The delivery device 102 can detect exposure of the contacts to the moisture based on, for example, changes in one or more electrical characteristics of the contacts. The delivery device 102, responsive to detecting the exposure of the contacts to the moisture, can initiate a mechanism (e.g., inflation of a balloon) that can, for example, appropriately orient the assembly 112 in relation to the delivery site 110.

The first communication device 104 and/or the second communication device 106 can be positioned or located external to the subject, and each can be capable of communicating with the delivery device 102, and can be capable of communicating with each other.

The first communication device 104 can include a first communication device transmitter 130, a first communication device receiver 132, a first communication device memory device 134, and/or a first communication device controller 136. The second communication device 106 may be structured with components similar to those of the first communication device 104 (e.g., the second communication device 106 may include a transmitter, receiver, memory device, and controller), or may be structured with additional, fewer, or different components than the first communication device 104.

The first communication device 104 can be used to communicate with the delivery device 102 via transmittal from the transmitter 130 of the first communication device 104 to the receiver 124 of the delivery device 102, and/or via reception by the receiver 132 of the first communication device 104 from the transmitter 122 of the delivery device 102. Similarly, the second communication device 106 can be used to communicate with the delivery device 102.

The first communication device 104 can be used to communicate with the second communication device 106. Similarly, the second communication device 106 can be used to communicate with the first communication device 104.

The memory device 134 of the first communication device 104 can store data received from the delivery device 102 and/or from the second communication device 106.

In an example communication scenario, the assembly 112 (e.g., via transmitter 122 and an antenna) can communicate data collected and/or generated by the detector 120 and/or the sensor(s) 140 to the first communication device 104 and/or to the second communication device 106, and can receive data and/or power from the first communication device 104 and/or from the second communication device 106. The data received by the delivery device 102 can include commands or instructions to execute one or more actions, such as activating the launch mechanism 118. In an embodiment, the first communication device 104 can be positioned in close proximity to the subject, and can provide power to the delivery device 102 through electromagnetic induction. The first communication device 104 also can communicate with the delivery device 102 (e.g., via low power and/or short-range communications). In an embodiment, the first communication device 104 can relay data received from the delivery device 102 to the second communication device 106. In another embodiment, the second communication device 106 can communicate directly with the delivery device 102. The first communication device 104 and/or the second communication device 106 can communicate with a cloud database 108. For example, the first communication device 104 and/or the second communication device 106 can relay or communicate data received from the delivery device 102 to the cloud database 108 (e.g., for storage, logging, processing and/or analysis).

In an embodiment, the delivery device 102 includes one or more passive or active RF identification (RFID) devices; the first communication device 104 and/or the second communication device 106 can power and read the RFID(s) to identify various components of the delivery device 102 and/or to read a status provided by one or more of the RFID(s).

The first communication device controller 136 can be programmed to control the operation of the various components of the first communication device 104.

The delivery device 102, the assembly 112, and the launch mechanism 118 can take many forms. FIGS. 2A, 2B, 3A, 3B, 4A, 4B, 5 and 6 illustrate examples of a cylindrical or other elongated and at least partially hollow structure of a chamber of the assembly 112 for which the launch mechanism 118 is a piston-like mechanism. In FIG. 2A and FIG. 2B, the piston-like mechanism is indicated as a piston 232 that deploys the carrier 114 by releasing (e.g., expelling or forcing) the carrier 114 out of the assembly 112. The carrier 114 contains the payload 116 (or is itself the payload 116). In an embodiment, the carrier 114 is retained within the delivery device 102 and the carrier 114 releases the payload 116 at or after deployment by the launch mechanism 118 to a delivery site; in another embodiment, the carrier 114 is itself delivered in the deployment by the launch mechanism 118 and the carrier 114 releases the payload 116 after deployment at a delivery site (or, the carrier 114 is itself the payload 116). In the examples of FIG. 2A-FIG. 2B, the carrier 114 has a pointed end for penetrating tissue.

The piston 232 includes a rod 234 and a base 236. One end of the rod 234 can be attached to and/or operates as a support or holder for the carrier 114, while the opposite end of the rod 234 can be attached to or supported by the base 236.

The assembly 112 includes a lower wall 224, an upper wall 226, and sidewalls 228 that extend between the lower wall 224 and the upper wall 226. The assembly 112 includes a breach surface 230. In the examples of FIG. 2A-FIG. 2B, the breach surface 230 sits above the upper wall 226; in another embodiment, the breach surface 230 is coplanar with the upper wall 226 and forms a portion of the upper wall 226; in another embodiment, the breach surface 230 sits below the upper wall 226. The breach surface 230 defines an opening through which the carrier 114 is deployed. In an embodiment, the breach surface 230 includes a portion that can be designed to break, puncture, tear, rip or disintegrate in response to a force exerted on it by the carrier 114; in an embodiment, the portion is a separate component attached to the breach surface 230 or to another surface of the assembly 112.

The assembly 112 in the examples of FIG. 2A-FIG. 2B can include or correspond to an encapsulated device for encapsulating the carrier 114. In an embodiment, the carrier 114 is fully encapsulated by the assembly 112, and the assembly 112 is sealed to maintain a sterile or aseptic environment within the assembly 112 until the carrier 114 breaches the breach surface 230. In such embodiments, the carrier 114 is introduced into the assembly 112, and the lower wall 224 and the upper wall 226 are both sealed, within an aseptic environment to create and maintain the aseptic environment within the assembly 112.

The sidewalls 228 define a channel 240 or guiding structure within which the base 236 can move in a direction along a longitudinal axis 238 of the carrier 114. The movement of the base 236 can be provided by a mechanical or propulsive force to the base 236. The lower wall 224 of the assembly 112, for example, can define an opening or an aperture (not shown) through which the mechanical or propulsive force can be provided to the base 236, causing the base 236 to move away from the lower wall 224. The channel 240 can guide the base 236 in a direction that is along the longitudinal axis 238 of the carrier 114 and towards the upper wall 226. A sustained force to the base 236 can cause the base 236 to move through the channel 240 such that the carrier 114 breaches through the breach surface 230 and at least partially exits the assembly 112.

Movement or position of the base 236 or the carrier 114 or breach of the carrier 114 through the breach surface 230 (any of which are referred to in general herein for convenience as deployment of the carrier 114) can be detected by the detector 120 (FIG. 1).

In an embodiment, responsive to detecting deployment of the carrier 114, the detector 120 can trigger or cause the controller 128 to send (via the transmitter 122) one or more signals to the first communication device 104 and/or the second communication device 106. An application (e.g., program or agent) executing on the first communication device 104 and/or the second communication device 106 may receive the one or more signals (e.g., via the first communication device receiver 132). In an embodiment, the application may record or store a timestamp, such as a date and/or a time of deployment of the carrier 114 (e.g., delivery of the payload 116).

In an embodiment, the first communication device 104 and/or the second communication device 106 may transmit received information to the cloud database 108. During clinical trials, for example, knowing the approximate or specific time of drug delivery can be helpful in pharmacokinetic studies with short half-life drugs (e.g., to indicate a starting point for blood analysis); or, during therapeutic treatment, knowing the approximate or specific time of drug delivery can be helpful to determine an expected amount of a component of a therapeutic formulation in the bloodstream over time for a single dose or multiple sequential doses. In addition, the cloud database 108 can collect and/or collate data from one or more subjects. Such information collected via the application(s) and/or the cloud database 108 can be used, for example, in post-market monitoring of a subject, during studies (e.g., Phase IV studies), or to collect usage information over a population of subjects. If it is detected that the subject is late or past a schedule for completion of drug delivery (e.g., forgets to take a capsule or pill incorporating the delivery device 102), the application can remind the subject via the first communication device 104 and/or the second communication device 106, hence facilitating and improving compliance by the subject.

The controller 128 can also send information associated with each delivery of the payload 116, and information for traceability and/or compliance purposes. The information (e.g., for traceability and/or compliance purposes) can help avoid or track an expired or expiring lot, and help identify manufacturing concerns by lot number.

In an embodiment, the controller 128 can detect, collect and/or store parameter profiles (e.g., deployment, motion, location, orientation, temperature, pressure, pH, and/or gas flow information) over one or more time instances, to internal memory (e.g., the memory device 126), and can transmit the information as well as other data (e.g., type of therapeutic formulation, part number, lot number) to the application of the first communication device 104 and/or the second communication device 106. Such information can be further relayed to or stored at the cloud database 108. This information can also be used to initiate proper delivery of the payload 116, by allowing the application or user to identify suitable conditions and a time instance to remotely trigger deployment of the carrier 114 via a signal from the first communication device 104 and/or the second communication device 106. This information can be monitored before, during, and after delivery of the payload 116, to check for or ensure proper delivery of the payload 116. This information can be used to monitor compliance to a therapy regimen and schedule (e.g., to ensure that a capsule is swallowed), the required schedule is adhered to (e.g., via recorded timestamps), and the proper therapeutic preparation is administered (e.g., via transmission of pre-loaded part number or lot number).

In an embodiment, the stored and/or transmitted information can be used to characterize a target region (e.g., the GI tract). For example, in an embodiment in which the delivery device 102 is a capsule containing a deployable balloon and an embodiment of the assembly 112, the information can include a log of ambient temperature (e.g., inside and/or outside the capsule, inside and/or outside the balloon), ambient pressure (e.g., inside and/or outside the capsule, inside and/or outside the balloon), applied forces (e.g., on the capsule, on the balloon), pH (e.g., inside and/or outside the capsule, inside and/or outside the balloon), orientation and/or motion data (e.g., of the capsule, balloon, chamber, carrier) such as velocity, acceleration, gyroscopic conditions (e.g., yaw, pitch, roll), or video data.

In an embodiment, the stored and/or transmitted information can be used to perform one or both of diagnostics and therapy. For example, the information can be used to detect a previously-applied marker (e.g., ink or other dye, radiopaque mark, metal staple, metal plate, or metal piece) or implant. The information can be also used to monitor surroundings of such implant or marker. For example, the information can be used to detect indications of swelling or infection, identify the marker, and deliver the payload 116 (e.g., the therapeutic preparation or electronics) at the marker. The information can further be used to compare the GI tract data of the subject to previously-acquired characteristic data for that subject to monitor for changes. For example, information on such changes may be used to titrate or otherwise adjust (e.g., cease) the delivery of a given therapeutic preparation.

The information can be used to compare the GI tract data of the subject to previously-acquired characteristic data for a patient population with respect to a certain condition, to identify whether the subject has a likelihood of that condition (e.g., by training a machine learning model and/or using a machine learning model). The information can be used to compare the GI tract data of the subject to previously-acquired characteristic data of a patient population in general, to identify potential anomalies (e.g., by using a trained machine learning model).

In an embodiment, the information can be used to detect the presence of a chemical or other substance, including one or more metabolic analytes and/or biomarkers (e.g., insulin, glucose, partial pressure of oxygen (PO₂), hemoglobin (including oxy and deoxy hemoglobin), iron, glucose, bile, or cholesterol) as well as cells indicative of a disease or condition, or indicative of a change in such a disease or condition.

In an embodiment, the information can be used to detect presence of temperature anomalies (e.g., high temperature relative to body temperature), or high variations in temperature, or other characteristic.

Information such as parameter profile(s) can correspond to reporting of various characteristics and/or occurrences. Such information can be used to determine proper or successful delivery of therapeutic preparation to the subject, including proper operation of a delivery device (e.g., the delivery device 102) as expected, confirmation of a location of the device in the GI tract, and/or location of a delivered therapeutic preparation (e.g., into an intestinal wall, into a peritoneal cavity).

Attention is now directed to the specific examples illustrated in FIG. 2A-FIG. 2B.

FIG. 2A illustrates a cross-sectional view of an example of the assembly 112, including a detector 220 (e.g., an example of the detector 120 in FIG. 1). The detector 220 is structured to detect contact with, or proximity to, a defined portion 222 (e.g., a bump or contact point) of the assembly 112 indicative of deployment of the carrier 114. The detector 220 in the example of FIG. 2A is positioned on a surface of the base 236 that faces the upper wall 226, such that movement of the base 236 within the assembly 112 causes the detector 220 to move closer to the defined portion 222. The defined portion 222 in the example of FIG. 2A is positioned on an inner surface of the upper wall 226 and opposite the position of the detector 220.

During deployment, the movement of the base 236 towards the upper wall 226 can cause a reduction in a distance or separation between the detector 220 and the defined portion 222. In an embodiment, deployment of the carrier 114 can be defined as a position of the base 236 where the base 236 is in close proximity to or in contact with the upper wall 226. For example, when a propulsive force is applied to the base 236, the base 236 moves towards the upper wall 226. The continuous application of the propulsive force can cause the base 236 to continue to move toward the upper wall 226 until the upper wall 226 (or another impediment provided by a design of the assembly 112) causes the base 236 to stop moving. At this position, the proximity and/or contact of the detector 220 and the defined portion 222 can be communicated to the controller 128; for example, one or both of the detector 220 and the defined portion 222 can include conductors such that they form a portion of a detection circuitry in electrical communication with the controller 128. The detection circuitry can detect the proximity or contact of the detector 220 and the defined portion 222 by way of a change in a received voltage or current, or by a switching mechanism. For instance, the detector 220 can include an electrical switch (e.g., an electric contact switch), an electro-mechanical sensor (e.g., including a piezoelectric transducer, limit switch, actuator, or a resistive sensor), a pneumatic sensor, a magnetic sensor, an inductive sensor (e.g., an inductive proximity switch), a capacitive sensor, a photoelectric sensor (e.g., a diffuse sensor), a reflective sensor, a through-beam sensor, an infrared (IR) photodetector, or an ultrasonic sensor (e.g., using surface acoustic wave absorption).

In an embodiment, deployment of the carrier 114 can be defined as a position of the base 236 intermediate to a position that is nearest to the lower wall 224 and a position that is nearest to the upper wall 226. In an embodiment, the detector 220 is a proximity detector that detects the proximity of the detector 220 to the defined portion 222 in comparison to a threshold value. The proximity detector can be a magnet sensor, a capacitive sensor, an inductive sensor, an optical sensor, a photoelectric sensor, an ultrasonic sensor, a diffuse sensor, a reflective sensor, a through-beam sensor, or any other proximity measuring or detecting sensor. The controller 128 can be structured to measure an appropriate parameter to determine whether the detector 220 is in contact with or in close proximity to the defined portion 222. The position of the detector 220 can be different from that shown in FIG. 2A. For example, the detector 220 can be positioned on the rod 234 or the carrier 114, instead of being positioned on the base 236. The defined portion 222 can be appropriately positioned on the assembly 112 to allow for contact or proximity detection associated with deployment of the carrier 114.

FIG. 2B illustrates a cross-sectional view of an example of the assembly 112 that includes a detector 260 (e.g., an example of the detector 120 in FIG. 1), alternatively or additionally to the detector 220 in FIG. 2A. The detector 260 can include a trigger 262 and a switch 264. The trigger 262 can be a moveable member (e.g., a trigger pin or button), which when pressed on activates the switch 264. In an embodiment, the trigger 262 can be spring loaded such that some portion of the trigger 262 can protrude from a surface of the base 236. During deployment, a force (e.g., a propulsive force) can move the base 236 in a direction along the longitudinal axis 238 of the carrier 114. This can cause the base 236 to move towards the upper wall 226 of the assembly 112. The motion of the base 236 eventually positions the base 236 near the upper wall 226, and the upper wall 226 pushes against the trigger 262, which activates the switch 264, indicating deployment of the carrier 114.

In an embodiment, the detection circuitry can include sensor(s) for the voltage, the current, or both at the switch 264 to detect activation of the switch 264. In an embodiment, the switch 264 or the detection circuitry can be connected to an input-output (I/O) port of the controller 128, and the controller 128 can detect the activation of the switch 264.

The trigger 262 can be shaped and/or positioned anywhere on the base 236 that can allow the trigger 262 to move or be initiated when the base 236 is in a position that corresponds to deployment of the carrier 114. As an example, the trigger 262 may include a trigger pin. When the carrier 114 is deployed to the top of the assembly 112, the trigger pin may come in contact with the upper wall 226 of the assembly 112, and can be pushed through the base 236 by continued momentum of the base 236 towards the upper wall 226. The trigger pin, when pushed through (or relative to) the base 236, can activate the switch 264 which indicates deployment of the carrier 114.

FIG. 2C illustrates an example detector 270 that can be used to implement the detector 260 discussed above in relation to FIG. 2B. The detector 270 can include a trigger 272, which can be similar to the trigger 262 discussed above in relation to FIG. 2B. The trigger 272 can be attached to a switch 274. In particular, the trigger 272 can be disposed on one end of an electrically conductive bridge 276, the other end of which can be attached to (or integrally formed with) a first contact surface 278 disposed on a top surface of the switch 274. A second contact surface 280 may be positioned on the top surface of the switch 274 below the end of the conductive bridge 276 on which the trigger 272 is positioned. The second contact surface 280 and the first contact surface 278 may be separated by an insulating material. Optionally, a standoff 290 can be positioned between the first contact surface 278 and the second contact surface 280 and also between the conductive bridge 276 and the top surface of the switch 274. When the trigger 272 is pressed towards the top surface of the switch 274, the conductive bridge 276 can bend downwards, or if the standoff 290 is made of flexible material compresses the standoff 290. With sufficient force on the trigger 272, the end of the conductive bridge 276 near the trigger 272 can make contact with the second contact surface 280, causing a conductive path between the first contact surface 278 and the second contact surface 280. The formation of this conductive path can be detected to indicate deployment of the carrier 114.

In an embodiment, instead of (or in addition to) the standoff 290, the conductive bridge 276 can include memory metal that has a memory in the non-contact state. In a normal or non-contact state, the trigger 272 can be positioned on one end of the electrically conductive bridge 276, the other end of which can be attached to the first contact surface 278 of the switch 274. The second contact surface 280 may be positioned on the top surface of the switch 274 below the end of the conductive bridge 276 on which the trigger 272 is positioned. The second contact surface 280 and the first contact surface 278 may be separated by an insulating material. When the trigger 272 is pressed towards the top surface of the switch 274, the conductive bridge 276 can bend downwards (e.g., against the memory of the memory metal). With sufficient force on the trigger 272, the end of the conductive bridge 276 near the trigger 272 can make contact with the second contact surface 280, causing a conductive path between the first contact surface 278 and the second contact surface 280.

In an embodiment, the switch 274 can be implemented using, or can be replaced by, one or more of an electrical switch, a piezoelectric switch, a capacitive transducer, a resistive transducer, and/or other type of switch discussed herein.

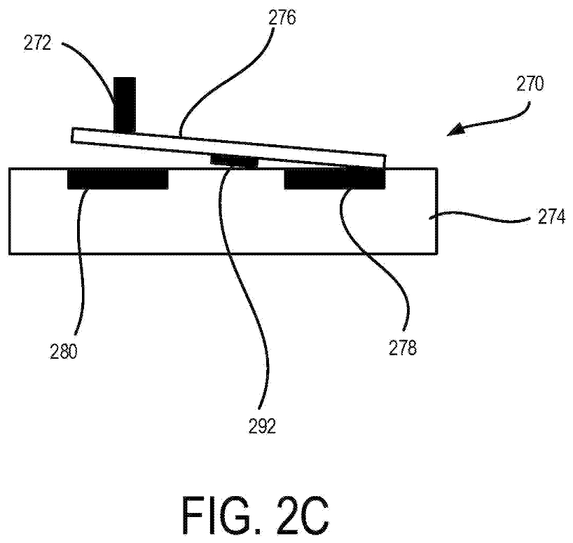

FIG. 3A illustrates a cross-sectional view of an example of a launch assembly 300 including a carrier 302 coupled to a piston 304. The assembly 300 includes a stud 306 integral with or attached to a remainder of the piston 304. A set of electronics 308 (e.g., incorporated on a printed circuit board) is positioned adjacent to the piston 304. A detector 310 is coupled to the set of electronics 308. The detector 310 includes a conductive bridge 312 electrically coupled to the set of electronics 308 on one end, and the detector 310 further includes a contact surface 314. The stud 306 extends through the set of electronics 308 and pushes against the conductive bridge 312 prior to deployment of the piston 304 and thus the carrier 302 to keep the conductive bridge 312 from contacting the contact surface 314.

FIG. 3B illustrates the launch assembly 300 of FIG. 3A after the piston 304 starts to deploy. The piston 304 moves away from the set of electronics 308, such that the stud 306 pulls away from the set of electronics 308, allowing the conductive bridge 312 to make electrical contact with the contact surface 314.

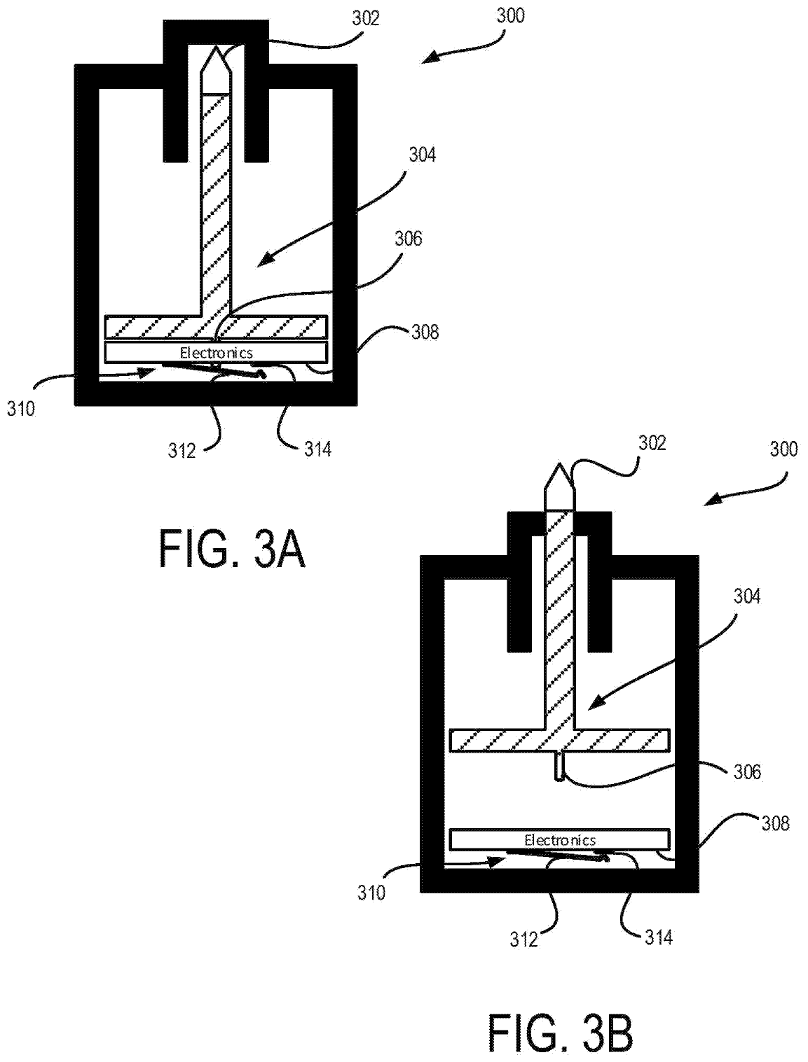

FIG. 4A illustrates a cross-sectional view of an example of a launch assembly 400 including a carrier 402 coupled to a piston 404. The assembly 400 includes a stud 406 attached to a remainder of the piston 404 by a wire or string 405. A set of electronics 408 (e.g., incorporated on a printed circuit board) is positioned adjacent to the piston 404. A detector 410 is coupled to the set of electronics 408. The detector 410 includes a conductive bridge 412 electrically coupled to the set of electronics 408 on one end, and the detector 410 further includes a contact surface 414. The stud 406 extends through the set of electronics 408 and pushes against the conductive bridge 412 prior to deployment of the piston 404 and thus the carrier 402 to keep the conductive bridge 412 from contacting the contact surface 414.

FIG. 4B illustrates the launch assembly 400 of FIG. 4A after the piston 404 starts to deploy. The piston 404 moves away from the set of electronics 408, such that the piston 404 pulls the string 405 which then pulls the stud 406 away from the set of electronics 408, allowing the conductive bridge 412 to make electrical contact with the contact surface 414.

FIG. 4C illustrates a frame 450 attached to an expandable extensor 420 which is coupled to a stud 430. The frame 450 is attached, for example, to a piston (e.g., the piston 404). Similar to the illustration in FIG. 4A and FIG. 4B, the stud 430 can extend through a structure (e.g., the electronics 408). The extensor 420 replaces the string 405 in FIG. 4A and FIG. 4B, such that when the piston moves, the frame 450 (which is attached to the piston) moves and the extensor 420 expands. Once the piston has moved a ways, the extensor 420 pulls the stud 430 to allow a detector (e.g., the detector 410) to detect deployment of the piston and thus the carrier.

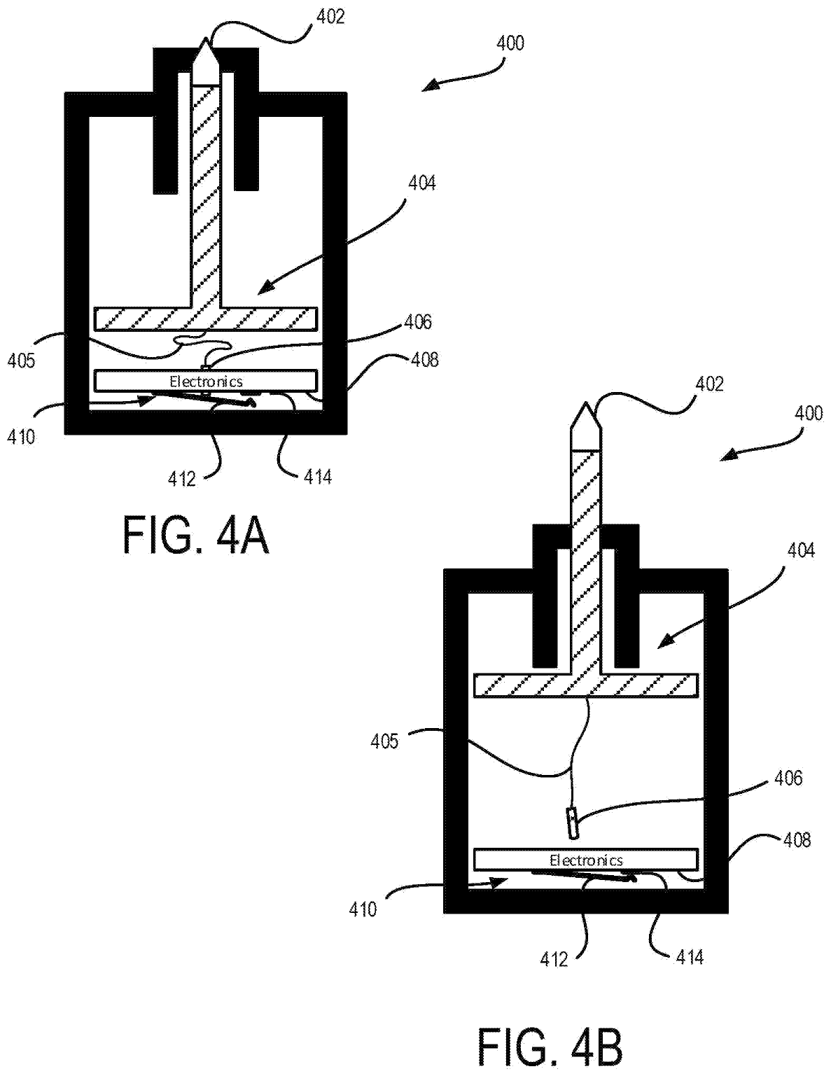

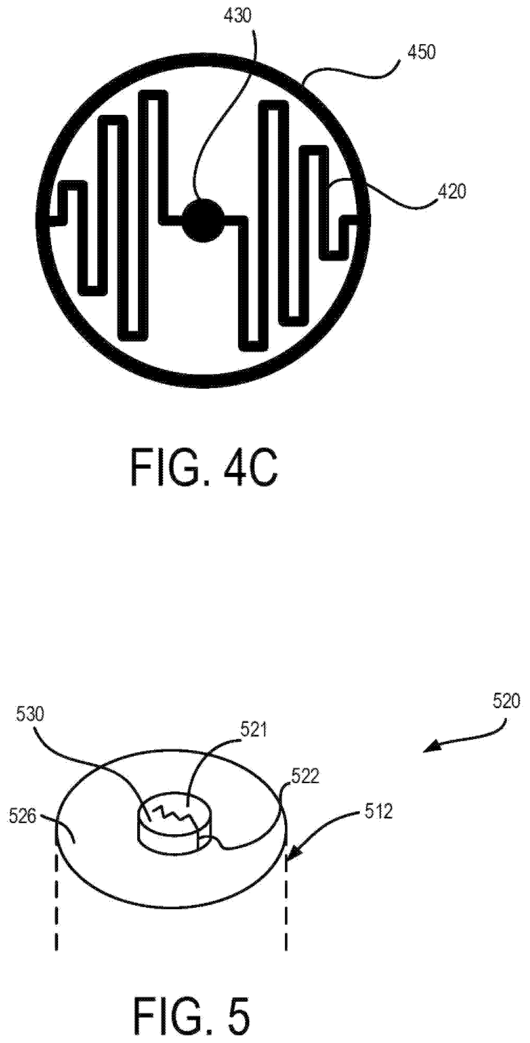

FIG. 5 depicts a detector 520 (e.g., an example of the detector 120 in FIG. 1) that can be used in conjunction with the embodiments illustrated in FIG. 1-FIG. 4B, FIG. 6 or other embodiments. In particular, the detector 520 can include a material 521 positioned on a breach surface 530 of the assembly 112. In an embodiment, the carrier 114 is pushed through the material 521, and a detection circuitry detects disruption or breach of the material 521 and causes a signal to be sent to the controller 128.

In an embodiment, the material 521 can be made of a thin layer of metal or alloy (e.g., a thin layer of aluminum foil) or other conductive material that can be breached (e.g., torn, disrupted, disintegrated, or perforated) when the carrier 114 is deployed. In an embodiment, the material 521 is a conductive or non-conductive material incorporating one or more conductive wires, traces, fibers, threads, or inks that can be broken when the carrier 114 is deployed.

One or more wires 522 can be connected to the material 521 such that the material 521 and the wires form a portion of detection circuitry of the delivery device 102 to detect a breach of the material 521 by detecting a change in one or more electrical characteristics of the material 521, such as detection of a circuit break due to the carrier 114 tearing through or disrupting the material 521 or breaking the conductive wires, traces, fibers, threads, or inks incorporated in the material 521. The breach of the material 521 can result in a change in one or more electrical characteristics of the material 521. The detection circuitry can be structured to detect these changes. For example, the breach of the material 521 can break a circuit or increase the resistance of the material 521, which change can be detected by the detection circuitry, such as by the controller 128.

In an embodiment, the detection circuitry can compare the measured electrical characteristic of the material 521 with a characteristic curve (e.g., across one or more lots of the material 521 and/or one or more lots of the assembly 112). In certain embodiments, the detection circuitry can compare the measured electrical characteristic of the material 521 with a characteristic curve which can have values corresponding to various states of the material 521. The various states can include, for example: no deployment of the carrier 114 (e.g., intact material 521), partial deployment of the carrier 114, and/or deployment of the carrier 114 (e.g., material 521 fully breached). Comparison of the measured value with the value on the characteristic curve can indicate the state of the material 521. The electrical characteristic of the material 521 measured by the detection circuitry can be, for example, a resistance, a capacitance, or an inductance of the material 521. Multiple such electrical characteristics may be measured by the detection circuitry.

In an embodiment, the wires 522 can be painted or printed on the breach surface 530. In an embodiment, the wires 522 can be laid over or under the breach surface. In an embodiment, all, or a portion of, the detection circuitry (e.g., including the wires 522) can be located or embedded in a neck region of the assembly 112 defined by sidewalls that extend between the breach surface 530 and an upper wall 526. Multiple wires 522, and/or multiple conductive wires, traces, fibers, threads, or inks, can be used to allow for more resolution in determining where a breach occurs in the material 521 (e.g., by using a ladder resistance circuit, or a matrix resistance circuit).

FIG. 6 illustrates a cross-sectional view of an embodiment of the assembly 112 having a lower wall 624, an upper wall 626, sidewalls 628, a breach surface 630, and defining a channel 640. The assembly 112 further includes a carrier 114, a piston 632 having a rod 634 and a base 636, each functioning similar to similarly-named components in FIG. 2A. The assembly 112 also includes a detector 620 (e.g., an embodiment of the detector 120 in FIG. 1).

In particular, the detector 620 can include an air flow or a gas flow detector that can detect a flow 622 of gas or air into the assembly 112. In an embodiment, the flow 622 into the assembly 112 is due to gas or air drawn or pulled into the assembly 112 when the base 636 moves through a trajectory for deployment of the carrier 114. In an embodiment, the flow 622 into the assembly 112 is used to provide a propulsive force to the base 636. The propulsive force can cause the base 636 to move in a direction along a longitudinal axis 638 of the carrier 114. A sustained propulsive force can cause the base 636 to continue its motion in the channel 640 and thereby cause the carrier 114 to breach the breach surface 630 of the assembly 112.

The detector 620 can include an air flow or gas flow sensor (e.g., located within the assembly 112), that determines a magnitude of air or gas flow (for example, in standard cubic centimeter per minute (SCCM) units) entering the assembly 112. The detector 620 may form a portion of a detection circuitry which can correlate the air flow measurement by the detector 620 with a predicted position or movement of the base 636 and/or the carrier 114. For example, the controller 128 (FIG. 1) can store a look-up table (or a transformation mathematical formula) corresponding to a characteristic curve which provides a relationship between various air flow values and the corresponding positions or movements (or end of movement at deployment) of the base 636. The characteristic curve, for example, can be experimentally determined, and/or can be generated using computer simulation. The detection circuitry, based on the air flow measurement value(s) from the detector 620, can determine deployment of the carrier 114. The detection circuitry can also monitor or take into consideration the air flow measurements from the detector 620 before, during, and/or after deployment of the carrier 114, to check, for example, for any leakage of air to the chamber before deployment, appropriate velocity of the base 636 during deployment, and/or for appropriate deflation air flow after deployment of the carrier 114. For example, the detection circuitry can compare the air flow measurement(s) from the detector 620 to one or more predetermined characteristic curves.

In an embodiment, other example detectors, alternatively or in addition to the ones discussed above in relation to FIGS. 2A-6, can be used. For example, in an embodiment, a velocity meter or an accelerometer can be used to determine deployment of the carrier 114. Detection circuitry can monitor an output from the velocity meter or accelerometer mounted on a base (e.g., the base 236 or the base 636) of a piston (e.g., the piston 232 or the piston 632) to determine a time, velocity, and/or acceleration at a beginning of the motion of the base due to the propulsive force and a time, velocity, and/or acceleration at the end of the motion of the base to determine whether time, velocity, and/or acceleration magnitude values corresponds to a known profile indicative of a successful deployment of the carrier 114. For example, if a time between start and stop of the base is similar to a previously determined time corresponding to a successful deployment of the carrier 114, the detection circuitry may determine that successful deployment of the carrier 114 has occurred. For another example, if a ratio of a time to reach a peak of a curve to a time to reach a predefined threshold lower than the peak is outside of a predetermined range for the ratio for that curve, the detection circuitry may determine that deployment of the carrier 114 was not successful.

In an embodiment, detectors can include electrical switches, electro-mechanical sensors, pneumatic sensors, magnetic sensors, inductive sensors, capacitive sensors, photoelectric sensors, or ultrasonic sensors. Measurements from one or more of these detectors can be used to determine the successful deployment of the carrier 114.

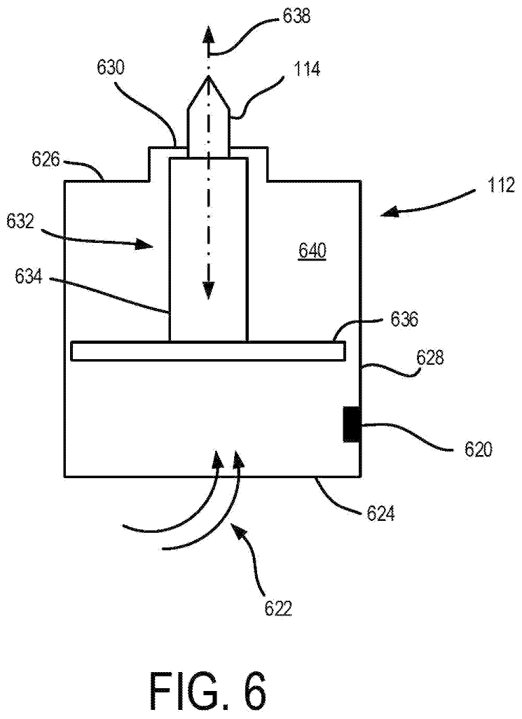

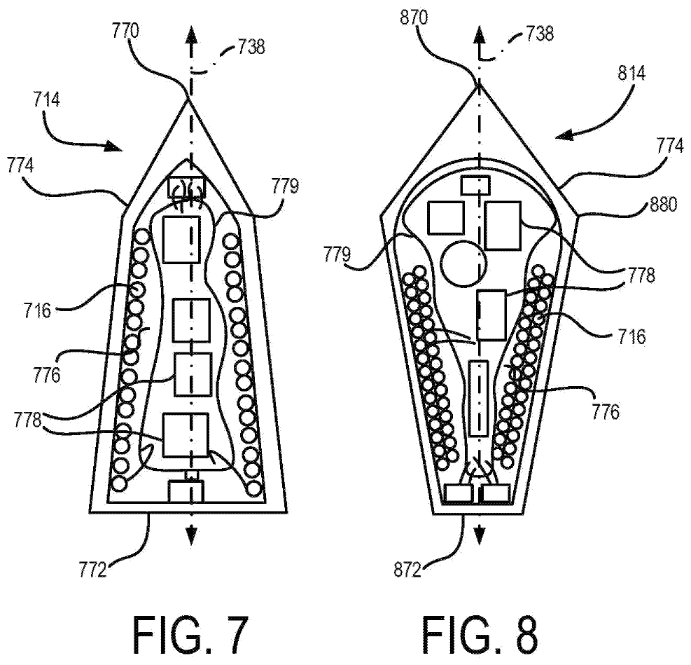

FIGS. 7 and 8 illustrate cross-sectional views of examples of the carrier 114 in which the carrier 114 contains a payload 116 of one or more electronic components, additional to or exclusive of a therapeutic preparation. In particular, FIG. 7 illustrates the cross-sectional view of a first example carrier 714 and FIG. 8 illustrates a cross-sectional view of a second example carrier 814. The carrier 714 and the carrier 814 are each illustrated as having a pointed end (e.g., to aid in penetration of the respective carrier into the delivery site 110) and sloping sides. In other embodiments of the carrier 114, the sides are not sloped, and/or both ends are pointed, and/or neither end is pointed. In general, a shape and dimensions of the carrier 114 can be structured for suitability to the target delivery site, suitability for the delivery technique, and/or suitability for the structure of the delivery device 102.

The carrier 714 has a first end 770 and a second end 772. The carrier 714 has a shape where the cross-sectional area, measured normal to a longitudinal axis 738 of the carrier 714, increases with an increase in distance from the first end 770. The carrier 714 can include a housing 774 that defines a cavity 776, which houses an antenna 716 (e.g., which may be a wire wrapped circularly within the cavity 776 such that ends of the wire appear as circles in the cross-section as illustrated) and electrical components 778, for example disposed on a circuit board 779.

The electrical components 778 can include sensors and/or a therapeutic preparation deployment mechanism. In an embodiment, the electrical components 778 can include a detector, such as for detecting deployment of the carrier 714 out of the assembly 112. For example, the carrier 714 can include detector(s) for determining a proximity or location of the carrier 714 relative to the assembly 112. This information may also be used in conjunction with information regarding one or more environmental conditions of the carrier 714 or the delivery device 102 (e.g., temperature, pressure, pH) in comparison with a characteristic or expected profile (e.g., a characteristic curve) or threshold, such as for determining an orientation or motion of the carrier 714 relative to the assembly 112. In an embodiment, the carrier 714 can include additional circuitry that, in response to detecting deployment of the carrier 714, can activate deployment of a therapeutic preparation included in the carrier 714 as part of the payload 116 into the delivery site 110 and/or activate transmission of data from within the carrier 714. The antenna 716 (in conjunction with communication electronics such as one or more of the electrical components 778) can be used to transmit data from the carrier 714 to the delivery device 102, and/or to the first communication device 104, and/or to the second communication device 106. The antenna 716 (in conjunction with communication electronics such as one or more of the electrical components 778) can further be used to receive information from the delivery device 102, the first communication device 104, and/or the second communication device 106; such as data from which the carrier 714 determines a time at which to release the therapeutic formulation from the carrier 714, or such as instructions to release the therapeutic formulation from the carrier 714.

The second example carrier 814 is similar to the first example carrier 714 in many respects, and like components have been labeled with like reference numerals. The carrier 814 includes a first end 870 and a second end 872. However, unlike the carrier 714, the cross-sectional area of the carrier 814 measured normal to the longitudinal axis 738 first increases with an increase in a distance from the first end 870 up to an intermediate portion 880, and then decreases past the intermediate portion 880. The carrier 814 can function in a manner similar to that discussed in relation to the carrier 714.

FIG. 7 and FIG. 8 illustrate examples of an embodiment of the carrier 114 incorporating electronics. In other embodiments, the carrier 114 does not incorporate electronics. In an embodiment, the carrier 114 incorporates electronics in addition to electronics incorporated elsewhere in the delivery device 102 (e.g., the detector 120, the transmitter 122, the receiver 124, the memory device 126, the controller 128, and/or the sensors 140). Accordingly, the carrier 114 can monitor its environment and its deployment, initiate release of the therapeutic formulation from the carrier 114 (if applicable), and communicate with the delivery device 102, the first communication device 104, and/or the second communication device 106.

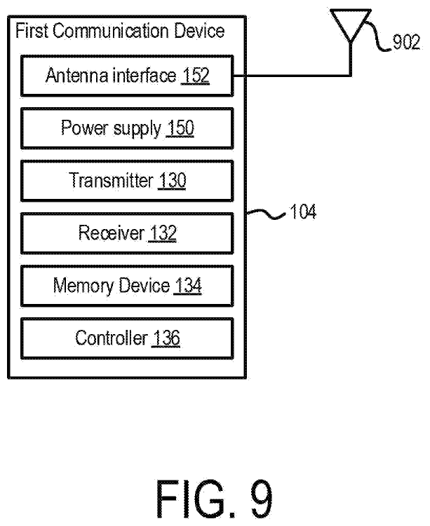

FIG. 9 illustrates a detailed block diagram of the first communication device 104 discussed above in relation to FIG. 1. As discussed above, the first communication device 104 can include the first communication device transmitter 130, the first communication device receiver 132, the first communication device memory device 134, and/or the first communication device controller 136. The first communication device 104 can include a first communication device power supply 150 and/or a first communication device antenna interface 152. The first communication device 104 can be used to receive data from the delivery device 102. For example, the controller 128 of the assembly 112 can detect deployment of the carrier 114, and, in response, can transmit a signal, using the transmitter 122, including data indicating deployment of the carrier 114. The signal can be received by an antenna 902 of the first communication device 104. The antenna interface 152 can include interface circuitry (e.g., impedance matching circuitry) that couples the antenna 902 to the first communication device receiver 132. The first communication device receiver 132 can amplify, filter, and otherwise process the signal to extract the data embedded in the signal. For example, the first communication device receiver 132 can include a low-noise analog or radio frequency (RF) front end amplifier, an analog or RF filter, a digital signal processor, and other signal processing circuitry. The first communication device receiver 132 can provide the data to the first communication device controller 136, which can store the data in the first communication device memory device 134 and/or display the data to the user. The first communication device receiver 132 can also receive signals from the second communication device 106. Received information can be provided to the first communication device controller 136, which can determine an action to be taken based on the data received from the second communication device 106. For example, the data may include instructions or commands to send instructions to the assembly 112 or the delivery device 102 to carry out an action, such as, for example, deployment of the carrier 114, or deployment of a balloon. The first communication device controller 136 can transmit the commands or instructions to the delivery device 102 via the first communication device transmitter 130.

In an embodiment, the first communication device 104 can further include a user control and notification module that includes I/O and peripheral devices such as keyboard and/or display screen (e.g., a touchscreen) for receiving user input and providing user notifications. The first communication device 104 can also include additional communication interfaces, such as a network interface module (e.g., Ethernet, Wi-Fi, Bluetooth, 2G, 3G, 4G, 5G) to communicate data to the cloud database 108 and/or to the second communication device 106. In an embodiment, the power supply 150 can include a battery, a battery charger, and/or a power management circuitry that provides power in the form of one or more voltage potentials or currents to various components of the first communication device 104.

Referring again to FIG. 1, the delivery device 102 and the first communication device 104 can communicate signals and data using one or more wireless communication protocols. For example, the communication protocols can include Bluetooth, Bluetooth low energy (BLE), ZigBee, Z-wave, 6LoWPAN, Thread, wireless local area network (WLAN) or wireless fidelity (Wi-Fi), WiFi-ah (HaLow), 2G, 3G, 4G, 5G, long-term evolution (LTE) (Cat 0, 1, 3, M1), narrowband-Internet of Things (NB-IoT), near field communications (NFC), RFID, SigFox, LoRaWAN, Ingenu, Weightless-N, Weightless-P, Weightless-W, ANT & ANT+, DigiMesh, MiWi, EnOcean, Dash7, wireless highway addressable remote transducer (WirelessHART), frequency shift keying (FSK), or on-off keying (OOK).

By way of an example, the electronics of the assembly 112 can include an active RFID device that includes the transmitter 122 with a power source (e.g., a battery such as a lithium-ion battery), so that the transmitter 122 can send information (e.g., detected and/or stored by the sensor(s) 140 and/or the detector 120) to the first communication device 104. In an embodiment, the assembly 112 can include a passive RFID device that has no battery. The passive RFID device can draw power from an RFID reader (e.g., the first communication device 104) which sends out electromagnetic waves that induce a current in the passive RFID device's antenna. By drawing power from the RFID reader, the passive RFID device can transmit information (e.g., detected and/or stored by the sensor(s) 140 and/or the detector 120) to the first communication device 104.

Various embodiments also contemplate use of external antennas and associated receiver devices that can receive signals from the transmitter 122. In an embodiment, such antennas and receiver devices may be structured to be wearable by the user, and the antenna and the first receiver device can be integrated into a wearable structure. In an embodiment, the wearable structure can include a stretchable belt or band worn around the waist, chest, wrist, or other portion of the body; the belt or band can be structured to allow for a substantially one-size-fits-all design. Such embodiments can be achieved through the use of elastic fabric and other elastic materials.

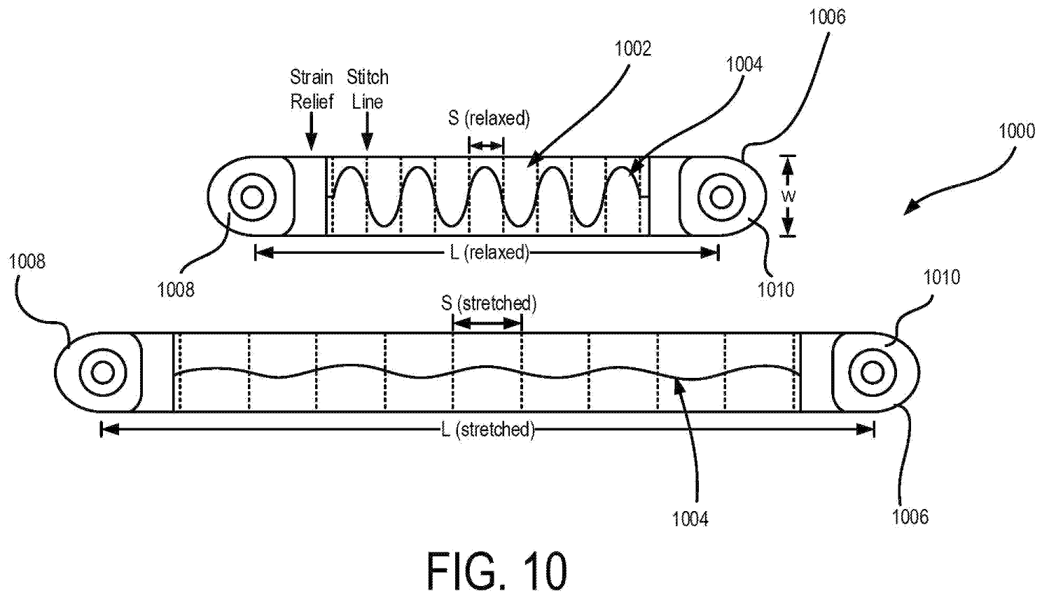

FIG. 10 illustrates an embodiment of a stretchable belt 1000 that can be used with the first communication device 104 shown in FIGS. 1 and 9 or other circuitry. FIG. 10 illustrates the belt 1000 in both a relaxed position (non-deployed) and a stretched (or deployed) position. A stretchable fabric 1002 provides both structure and stretchability for the belt 1000. In an embodiment, the belt 1000 is stretchable up to two or three times from its unstretched length, with even greater stretch capability contemplated. For example, in an embodiment the belt 1000 can have an unstretched length of about 28 inches which can be stretchable up to about 56 inches. Suitable stretchable fabrics include various polyether-polyurea copolymers, examples of which include Spandex or Lycra. The belt 1000 includes an antenna 1004 which may be stitched or otherwise attached to the fabric 1002 or other support material. In an embodiment, the antenna 1004 is stitched to the fabric 1002 at each stitch line, to substantially maintain a vertical position of a lengthwise axis of the antenna 1004 relative to a center line of the fabric 1002. In the embodiment shown, such a vertical position can be in the form of an approximately sinusoidal shape of the antenna 1004 relative to the centerline of the fabric 1002 and/or the belt 1000. When the fabric 1002 is stretched, the antenna 1004 is allowed to straighten out because it is not stitched into the fabric 1002 except at the stitch lines, which allows the lengthwise axis of the antenna 1004 to go from a vertical (e.g., sinusoidal) position relative to the center line of the fabric 1002 to a more linearly elongated position relative to the center line of the fabric 1002 and the belt 1000. Put in another way, the belt 1000 can have a contracted or unstretched state where the antenna 1004 is maintained in the aforementioned vertical (e.g., sinusoidal) position relative to the fabric 1002 or the belt 1000 centerline, and a stretched or expanded state (e.g., after the user puts the belt 1000 on and fastens the end portions) where the antenna has a linearly elongated orientation relative to the belt centerline. The belt 1000 can be worn by a subject around his/her waist, chest, or other portion of the body. The antenna 1004 can be coupled to a printed circuit board (PCB) or other circuitry which may include various electrical components (such as those discussed above in relation to FIG. 9). In an embodiment, the components included or associated with such PCB or other circuitry may correspond to one or more of a receiver, a processor, a digital signal processor, power management circuitry, user control and notification circuitry, a universal serial bus (USB) or other I/O port, a lithium ion or other battery, and/or a battery charger. It is to be understood that processors and other circuitry may include instructions, such as hard-wired instructions, firmware, or software, for controlling the processor or other circuitry in a desired fashion.

In an embodiment, optionally a PCB 1006 is attached at an end portion 1008 or 1010 of the belt 1000. In an embodiment, the PCB 1006 or other circuitry may be attached in other locations along the belt 1000 such as at central portion of the belt, or the belt 1000 may be attached to connections on the first communication device 104. The PCB 1006 provides a location to house electronics and/or battery, or provides an electrical connection between the antenna 1104 and the first communication device 104.

In the deployed or stretched position, the belt 1000 can be worn by the subject by coupling the end portions 1008, 1010 to the first communication device 104, or by coupling the end portion 1008 to the end portion 1010 of the belt 1000 (e.g., where the first communication device 104 is integrated with the belt 1000). End portions 1008 and 1010 may include a magnetic buckle or other fastening means so as to allow the user to easily buckle or fasten the end portions 1008 and 1010 together. During operation, the antenna 1004 can receive signals from the assembly 112 or the delivery device 102 and such signals can be provided to the first communication device 104 or other circuitry of the belt 1000. In an embodiment, components (e.g., the first communication device 104 or other circuitry) on the belt 1000 can be structured to process the signals to extract data embedded in the signals. In these and other embodiments, such components can also be structured to process the data and store the data in memory or relay the data to the second communication device 106 (e.g., a smartphone, tablet computing device, laptop, or other computing device) and/or to the cloud database 108. In use, embodiments of the belt 1000 allow the user to easily and unobtrusively receive signals from the transmitter 122, such as regarding the status of deployment of the carrier 114.

The belt 1000 is discussed herein as illustrative, and is by no means limiting in any way. Many other embodiments are also contemplated. For example, the first communication device 104 can be or be included as part of any type or form of device worn on the subject, such as on a lanyard or necklace (e.g., as a pendant device), an arm band (e.g., which may include or be incorporated into a fitness or health tracker device, or smartphone), a wristband (e.g., such as a smartwatch or exercise monitoring device), or attached to any type of belt. For example, an antenna can be implemented in any of the foregoing lanyard, necklace, arm band, wristband, belt or strap, and electrically coupled to the first communication device 104. As further examples, the first communication device 104 can include any type or form of device (e.g., smartphone, tablet computer, or the like) carried by the subject, for instance in a pocket, pouch, bag, or backpack. In additional or alternative embodiments, the first communication device 104 can include any type or form of device located proximate to the subject (e.g., connected to or in a vehicle, seat, bed, machine or computer operated by, occupied by, or otherwise used by the subject). In an embodiment, the belt 1000 can be structured to be a replacement belt or strap that can be used with or coupled to an existing belt loop, belt buckle, or strap holder.

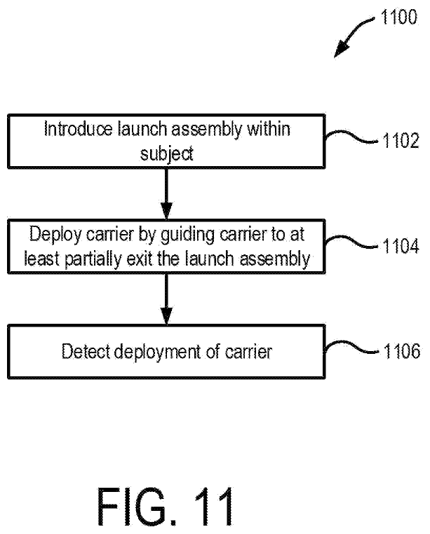

FIG. 11 illustrates a flow diagram of an example process 1100 for deploying a carrier (e.g., the carrier 114) in a subject and detecting carrier deployment. The process 1100 can be executed, for example, by the assembly 112 discussed above in relation to FIGS. 1-6. For example, the process 1100 can be executed by the controller 128. The process 1100 can include introducing (at 1102) a launch assembly (e.g., the launch assembly 112) within the subject. In an embodiment, the launch assembly can be part of a delivery device (e.g., the delivery device 102) that is orally ingested by the subject. The launch assembly can include the carrier that incorporates a payload (e.g., the payload 116), such as electronics and/or a therapeutic preparation.

The process 1100 can further include deploying (at 1104) the carrier by guiding the carrier (e.g., in a direction along a longitudinal axis of the carrier) to cause the carrier to at least partially exit the launch assembly. In an embodiment, a propulsive force can be provided to the launch assembly, which causes the carrier to move within a channel (e.g., the channel 240 or the channel 640) in a direction along a longitudinal axis of the carrier. In an embodiment, a continuous application of the propulsive force can cause the carrier to breach a penetrable portion (e.g., the breach surface 230 or the breach surface 630) of the launch assembly, which causes the carrier to partially or fully exit the launch assembly.

The process 1100 can further include detecting (at 1106) deployment of the carrier. In an embodiment, the launch assembly and/or the carrier include detectors that can detect deployment of the carrier. In an embodiment, the process 1100 includes the launch assembly detecting contact with or proximity to a defined portion of the launch assembly indicative of deployment of the carrier (e.g., using the detector 220, the detector 260, the detector 520, the detector 620, and/or other detectors). The determination that deployment has occurred may be relayed by the delivery device 102 to the first communication device 104 and/or the second communication device 106.

In an embodiment, the process 1100 includes the carrier detecting its own deployment, such as by detecting proximity to the launch assembly, or such as by detecting motion of the carrier; the proximity and/or motion over time may be compared to predefined characteristic curves stored in a memory of the carrier to identify when the carrier comes to rest after initiation of deployment. An example of such characteristic curve indicates an increasing motion from an initial value, crossing a predefined threshold value as motion increases, reaching a peak value of motion, decreasing motion, and crossing the threshold value or another predefined threshold value as motion decreases. Another example of such characteristic curve indicates an increasing motion from an initial value to a peak value followed by a decreasing motion, where a determination of deployment includes determining that motion has decreased by a predefined percentage of the peak value, or that a ratio of rise time to fall time of the motion curve reaches or crosses a predefined threshold ratio. Another example of such characteristic curve indicates an initial proximity to the launch assembly with a subsequent decreasing distance from the launch assembly to a predefined threshold value. In an embodiment, two or more determinations are made, such as determining that the carrier is remote from the launch assembly and determining that the carrier is no longer in motion. The determination that deployment has occurred may be relayed by the carrier to the delivery device 102, the first communication device 104, and/or the second communication device 106.

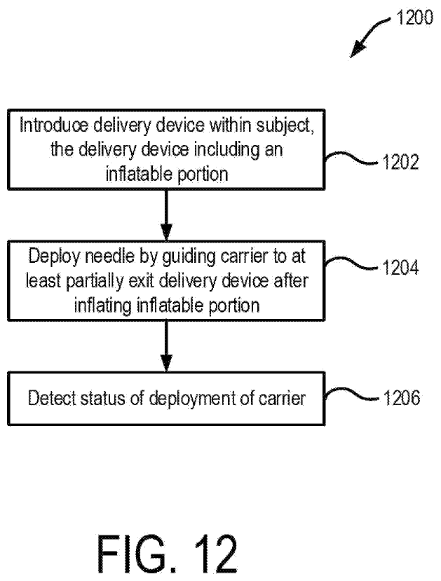

FIG. 12 illustrates a flow diagram of an example process 1200 for deploying a carrier (e.g., the carrier 114) in a subject and detecting carrier deployment. The process 1200 can be executed, for example, by the assembly 112 discussed above in relation to FIGS. 1-6. For example, the process 1200 can be executed by the controller 128. The second example process 1200 can include introducing (at 1202) a delivery device (e.g., the delivery device 102) within a subject, where the delivery device includes an inflatable portion that is coupled to a launch assembly.

The process 1200 can include guiding (at 1204) the carrier (e.g., in a direction along a longitudinal axis of the carrier) and deploying the carrier, after the inflatable portion of the delivery device is inflated to cause the carrier to at least partially exit the delivery device. In an embodiment, the inflatable portion of the delivery device can be inflated to position the launch assembly in proximity with a target delivery site (e.g., the delivery site 110). In an embodiment, once the inflatable portion of the delivery device is inflated, the carrier can be deployed by providing a propulsive force or other force within the delivery device. In an embodiment, the inflation of the inflatable portion provides the propulsive force.

The process 1200 can include detecting (at 1206) a status of deployment of the carrier. Detection can be, for example, as described with respect to FIG. 11 (at 1106).

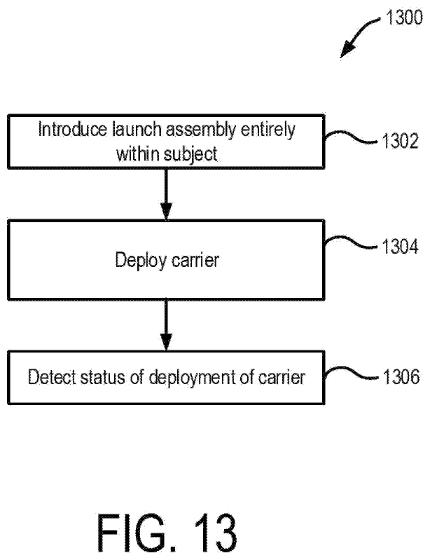

FIG. 13 illustrates a flow diagram of an example process 1300. The process 1300 can be executed, for example, by the assembly 112 discussed above in relation to FIGS. 1-6. For example, the process 1300 can be executed by the controller 128. The process 1300 can include introducing (at 1302) a launch assembly within the subject where the launch assembly includes detection circuitry and a carrier. As discussed above, the launch assembly can be orally ingested by the subject, and can make its way into the GI tract of the subject to arrive at a target region. The process 1300 can include deploying (at 1304), by the launch assembly, the carrier (e.g., in a direction along a longitudinal axis of the carrier). In an embodiment, a propulsive force can be provided to cause the carrier to move in a direction along a longitudinal axis of the carrier.

The process 1300 can include detecting (at 1306) a status of deployment of the carrier. Detection can be, for example, as described with respect to FIG. 11 (at 1106).

In an embodiment, the processes discussed above in relation to FIGS. 11-13 can include storing the status of deployment of the carrier in a memory device (e.g., the memory device 126), and/or transmitting the status to external devices, such as to the first communication device 104 and/or the second communication device 106. The processes can further include transmitting values of measurement from one or more sensors to the external devices. In an embodiment, the stored status and/or values of measurement are maintained in the internal memory device, and can be accessed from the memory device after the memory device is passed out of the subject. In certain embodiments, the stored status, values of measurement, and/or other information (e.g., for traceability) are maintained in the memory device, and can be queried, accessed and/or requested by an external device (e.g., the first communication device 104 and/or the second communication device 106). In an embodiment, queries can be made by an external device by querying an RFID device.

The processes can further include receiving commands or instructions from the external devices (e.g., to inflate the delivery device, or to launch the carrier). Responsive to receiving the command or instructions, the processes can include for instance initiating deployment of the carrier by applying a propulsive force within the launch assembly.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry (e.g., an FPGA or an ASIC).

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

Processors suitable for the execution of a computer program include, by way of example, both general and special purpose microprocessors, and any one or more processors of any kind of digital computer. Generally, a processor will receive instructions and data from a read-only memory or a random access memory or both. A computer includes a processor for performing actions in accordance with instructions and one or more memory devices for storing instructions and data. Generally, a computer will also include, or be operatively coupled to receive data from or transfer data to, or both, one or more mass storage devices for storing data (e.g., magnetic, magneto-optical disks, or optical disks). However, a computer need not have such devices. Moreover, a computer can be embedded in another device (e.g., a mobile telephone, a personal digital assistant (PDA), a mobile audio or video player, a game console, a Global Positioning System (GPS) receiver, or a portable storage device such as a USB flash drive). Devices suitable for storing computer program instructions and data (e.g., the memory device 126, the memory device 134, and on or more devices of the second communication device 106) include all forms of volatile or non-volatile memory, media and memory devices, including by way of example semiconductor memory devices (e.g., non-volatile memory such as EPROM, EEPROM, or flash memory devices, or volatile memory such as RAM, FRAM, MRAM, or DRAM); magnetic disks (e.g., internal hard disks or removable disks); magneto-optical disks; and CD-ROM and DVD-ROM disks. The processor and the memory can be supplemented by, or incorporated in, special purpose logic circuitry. The memory device 126 and the memory device 134 may each be implemented in a single device or across multiple devices, such that each of the memory device 126 and the memory device 134 can have one or more of any of the above-mentioned devices suitable for storing computer program instructions and data.

To provide for interaction with a user, implementations of the subject matter described in this specification can be implemented on a computer having a display device (e.g., a CRT (cathode ray tube), plasma, or LCD (liquid crystal display)) for displaying information to the user, a keyboard, and a pointing device (e.g., a mouse or a trackball) by which the user can provide input to the computer. Other kinds of devices can be used to provide for interaction with a user as well; for example, feedback provided to the user can include any form of sensory feedback (e.g., visual feedback, auditory feedback, or tactile feedback); and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component (e.g., as a data server), or that includes a middleware component (e.g., an application server), or that includes a front-end component (e.g., a client computer having a graphical user interface or a Web browser through which a user can interact with an implementation of the subject matter described in this specification), or any combination of one or more such back-end, middleware, or front-end components. The components of the system can be interconnected by any form or medium of digital data communication (e.g., a communication network). Examples of communication networks include a local area network (LAN) and a wide area network (WAN), an inter-network (e.g., the Internet), and peer-to-peer networks (e.g., ad hoc peer-to-peer networks).

Embodiments of the present disclosure include, without limitation, the following aspects:
A device is provided for administering a therapeutic preparation within a subject, such as a human or other mammal. The device includes a carrier incorporating the therapeutic preparation, a base coupled to the carrier, and a launch assembly structured to be introduced within the subject. The launch assembly contains the base, and the carrier prior to deployment of the carrier. The launch assembly guides the base and/or the carrier during deployment, in a direction along a longitudinal axis of the carrier, to cause the carrier to at least partially exit the launch assembly and administer the therapeutic preparation to the subject. The device further includes a detector structured to detect status of deployment of the carrier.
A device includes detection circuitry structured to detect a status of a deployment of a carrier within a subject, and a launch assembly structured to be introduced within the subject. The launch assembly incorporates the detection circuitry and the carrier. The launch assembly is structured to deploy the carrier by at least partially ejecting the carrier from the launch assembly in a direction along a longitudinal axis of the carrier, and to cause the detection circuitry to detect the status.
A device includes a carrier, a launch assembly, and detection circuitry. The launch assembly is structured to be introduced within the subject and to deploy the carrier by ejecting the carrier from the device and into the internal tissue of the subject. The detection circuitry is structured to detect a status of a deployment of the carrier.
A system includes a delivery device including a portion that is inflatable, the delivery device structured to be introduced within a subject. The delivery device includes a launch assembly. The launch assembly guides the carrier, in deploying the carrier after the inflatable portion of the delivery device is inflated, to cause the carrier to at least partially exit the delivery device. The delivery device further includes detection circuitry structured to detect a status of deployment of the carrier.
A system includes a delivery device that is structured to be orally introduced within a subject, a carrier disposed in the delivery device, a launch assembly, and a detection circuit. The launch assembly is structured to cause the carrier to exit the delivery device so as to penetrate into internal tissue of the subject. The detection circuitry is structured to detect a status of deployment of the carrier.
A method includes: introducing a launch assembly within a subject, the launch assembly containing a carrier that incorporates a therapeutic preparation, and a base coupled to the carrier; deploying the carrier by guiding at least one of the base or the carrier in a direction along a longitudinal axis of the carrier, to cause the carrier to at least partially exit the launch assembly and administer the therapeutic preparation to the subject; and detecting a status of deployment of the carrier.
A method includes: introducing a delivery device within a subject, the delivery device including an inflatable portion coupled to a launch assembly that has a carrier and a base coupled to the carrier; guiding at least one of the carrier or the base in a direction along a longitudinal axis of the carrier, in deploying the carrier after the inflatable portion of the delivery device is inflated, to cause the carrier to at least partially exit the delivery device; and detecting a status of deployment of the carrier.
A method includes: introducing a launch assembly within a subject, the launch assembly incorporating detection circuitry and a carrier; deploying, by the launch assembly, the carrier, by at least partially ejecting the carrier from the launch assembly in a direction along a longitudinal axis of the carrier; and detecting, by detection circuitry, a status of deployment of the carrier.
A method includes: introducing a launch assembly within a subject, the launch assembly coupled to a carrier that incorporates a therapeutic preparation; deploying, by the launch assembly, the carrier; and detecting a status of deployment of the carrier.

The foregoing aspects, and aspects of other embodiments, may include without limitation one or more of the following features:
A carrier is deployed at a selected tissue site, such as into a wall of the GI tract.
Multiple carriers are deployed by a delivery device.
A carrier is a needle.
A detector is structured to detect contact with or proximity to a defined portion of a launch assembly indicative of deployment of the carrier.
A detector corresponds to one or more of: an electrical switch, an electro-mechanical sensor, a pneumatic sensor, a magnetic sensor, an inductive sensor, a capacitive sensor, a photoelectric sensor, or an ultrasonic sensor.
A detector includes a first electrical contact, a second electrical contact, and an electrically-conductive bridge extending over the first electrical contact and the second electrical contact. The detector includes a trigger pin coupled to the electrically-conductive bridge, where the electrically-conductive bridge includes a memory conductor, or the detector further includes a standoff for the electrically-conductive bridge.
A launch assembly includes a penetrable portion to allow a carrier to at least partially exit the launch assembly, and a detector is structured to detect breakage of the penetrable portion by the carrier. The penetrable portion may be a foil.
A detector is structured to detect gas flow within a launch assembly or into the launch assembly.
A detector is structured to detect a characteristic of motion of a carrier and/or a base relative to a launch assembly.
A detector is incorporated to a carrier.
A detector can be structured to detect a variety of properties and characteristics (collectively referred to herein as characteristics) related to a device, a launch assembly, a carrier, or surroundings of any of them. Such characteristics may include, for example, one or more of: pressure, velocity, acceleration, orientation, temperature, pH, torsion on a wall of the launch assembly, tension on a penetrable portion of the launch assembly, vibration, gas flow, moisture, fluid conductivity, fluid chemistry, fluid composition, or location of the launch assembly.
A detector includes, or is coupled to, memory and transmission resources in order to store and transmit detected characteristic(s) to an external device.
An external device is worn on a subject, is held by the subject, or otherwise is in proximity to the subject.
A delivery device includes multiple detectors, each detecting similar characteristics or different characteristics.
A delivery device includes an inflatable device structured to align a launch assembly in a target position within a subject.
A delivery device includes a monitoring device structured to detect one or more predefined conditions, and to initiate deployment of a carrier responsive to detecting the one or more predefined conditions.
A delivery device includes a memory device structured to store information regarding deployment of a carrier and/or lot traceability information.
A method includes detecting contact with or proximity to a defined portion of a launch assembly indicative of deployment of a carrier.
A method includes detecting deployment of a carrier using at least one of: an electrical switch, an electro-mechanical sensor, a pneumatic sensor, a magnetic sensor, an inductive sensor, a capacitive sensor, a photoelectric sensor, or an ultrasonic sensor.
A method includes detecting deployment of a carrier using a detector that includes a first electrical contact, a second electrical contact, and an electrically-conductive bridge extending over the first electrical contact and the second electrical contact. The detector may further include a trigger pin coupled to the electrically-conductive bridge where the electrically-conductive bridge includes a memory conductor, or the detector further includes a standoff for the electrically-conductive bridge.
A method includes detecting breakage of a penetrable portion of a launch assembly by a carrier, the penetrable portion to allow the carrier to at least partially exit the launch assembly.
A method includes detecting gas flow within a launch assembly or into the launch assembly.
A method includes detecting a characteristic of motion of at least one of a carrier or a base relative to a launch assembly.
A method includes detecting, by detection circuitry incorporated to the carrier, movement and/or position of a carrier.
A method includes determining at least one of the following characteristics, for electronic storage or transmission: pressure, velocity, acceleration, orientation, temperature, pH, torsion on a wall of the launch assembly, tension on a penetrable portion of the launch assembly, vibration, gas flow, moisture, fluid conductivity, fluid chemistry, fluid composition, or location of the launch assembly.
A determined characteristic is transmitted to an external device which may be worn on, be held by, or otherwise be in proximity to, a subject.
A method includes aligning, by an inflatable device coupled to a launch assembly, the launch assembly in a target position within a subject.
A method includes detecting one or more predefined conditions, and initiating deployment of a carrier responsive to detecting the one or more predefined conditions.
A method includes storing in a memory device information regarding deployment of a carrier and/or lot traceability information.
A delivery device or method relates to a swallowable drug delivery device, such as, for example, for delivery of therapeutic preparations (alone or in formulations) into GI tissue.
A device is structured to propel a carrier to fully exit the device so as to pass into or through a wall of a gastrointestinal tract of a subject.
A detection circuitry is structured to detect contact with or proximity to a defined portion of a device indicative of deployment of a carrier.
A device comprises a penetrable portion to allow a carrier to exit the device, and detection circuitry structured to detect breakage of the penetrable portion.
A detection circuitry is structured to detect gas flow within a device.
A detection circuitry is structured to detect a characteristic of motion of at least one component of a device.
A detection circuitry is incorporated to a carrier.
A detection circuitry is structured to determine at least one of the following characteristics: pressure, velocity, acceleration, orientation, temperature, pH, torsion on a wall of the launch assembly, tension on a penetrable portion of the device, vibration, gas flow, moisture, fluid conductivity, fluid chemistry, fluid composition, or location of the launch assembly.
A detection circuitry is structured to transmit a determined characteristic to a communication device.
A transmitter is structured to transmit a signal including information on deployment status of a carrier.
A communication device is wearable by a subject.
A communication device includes a belt, a wrist band, a smart watch, or the communication device is incorporated into a smartwatch.
A detection is performed by a detection circuitry associated with a launch assembly.
A detection is performed by a detection circuitry associated with a carrier.
A detection is performed by a detection circuitry associated with a launch assembly and another detection is performed by a detection circuitry associated with a carrier.
At least one of the following characteristics is determined: pressure, velocity, acceleration, orientation, temperature, pH, torsion on a wall of the launch assembly, tension on a penetrable portion of the launch assembly, vibration, gas flow, moisture, fluid conductivity, fluid chemistry, fluid composition, or location of the launch assembly.
At least one characteristic is transmitted to an external communication device.
A deployment of a carrier is responsive to detecting one or more conditions.
A launch assembly is structured to deploy a carrier into or through: a wall of the stomach, a wall of the small intestine, a wall of the large intestine, or the peritoneal membrane.

Having now described some illustrative implementations, it is apparent that the foregoing is illustrative and not limiting, having been presented by way of example. In particular, although many of the examples presented herein involve specific combinations of method acts or system components, those acts and those components may be combined in other ways to accomplish the same objectives. Acts, components and features discussed only in connection with one implementation are not intended to be excluded from a similar role in other implementations or implementations.

While operations are depicted in the drawings in a particular order, this should not be understood as requiring that such operations be performed in the particular order shown or in sequential order, or that all illustrated operations be performed, to achieve desirable results. In some cases, the actions recited in the claims can be performed in a different order and still achieve desirable results. In addition, the processes depicted in the accompanying figures do not necessarily require the particular order shown, or sequential order, to achieve desirable results.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter. In one implementation, the systems and methods described herein consist of one, each combination of more than one, or all of the described acts or components.

Any references to implementations of components or acts of the systems and methods herein referred to in the singular may also embrace implementations including a plurality of these components, and any references in plural to any implementation or element or act herein may also embrace implementations including only a single element. References in the singular or plural form are not intended to limit the presently disclosed systems or methods, their components or acts, to single or plural configurations. References to any act or element being based on any information, act or element may include implementations where the act or element is based at least in part on any information, act, or element.

Where technical features in the drawings, detailed description or any claim are followed by reference signs, the reference signs have been included for the sole purpose of increasing the intelligibility of the drawings, detailed description, and claims. Accordingly, neither the reference signs nor their absence has any limiting effect on the scope of any claim elements.

The systems and methods described herein may be embodied in other specific forms without departing from the characteristics thereof. The foregoing implementations are illustrative rather than limiting of the described systems and methods. Also, components, characteristics, or acts from one embodiment can be readily recombined or substituted with one or more components, characteristics or acts from other embodiments to form numerous additional embodiments within the scope of the invention. Moreover, components that are shown or described as being combined with other components, can, in various embodiments, exist as standalone components. Further still, embodiments of the invention also contemplate the exclusion or negative recitation of an element, feature, therapeutic preparation, characteristic, value or step wherever said element, feature, chemical, therapeutic preparation, characteristic, value, step or the like is positively recited. Hence, the scope of the present invention is not limited to the specifics of the described embodiments, but is instead limited solely by the appended claims, and changes that come within the meaning and range of equivalency of the claims are embraced therein.

What is claimed is:

1. A device for delivering a therapeutic preparation to internal tissue of a subject, the device comprising:
a carrier comprising the therapeutic preparation;
a launch assembly structured to be introduced within the subject and to deploy the carrier by ejecting the carrier from the device and into the internal tissue of the subject; and
detection circuitry structured to detect a status of deployment of the carrier.

2. The device of claim 1, wherein the device is structured to propel the carrier to fully exit the device so as to pass into or through a wall of a gastrointestinal tract of the subject.

3. The device of claim 1, wherein the detection circuitry is structured to detect contact with or proximity to a defined portion of the device indicative of a deployment of the carrier.

4. The device of claim 1, wherein the device further comprises a penetrable portion structured to allow the carrier to exit the device, and the detection circuitry is structured to detect breakage of the penetrable portion.

5. The device of claim 1, wherein the detection circuitry is structured to detect gas flow within the device.

6. The device of claim 1, wherein the detection circuitry is structured to detect a characteristic of motion of at least one component of the device.

7. The device of claim 1, wherein the detection circuitry is incorporated to the carrier.

8. The device of claim 1, wherein the detection circuitry is structured to determine at least one of the following characteristics: pressure, velocity, acceleration, orientation, temperature, pH, torsion on a wall of the launch assembly, tension on a penetrable portion of the device, vibration, gas flow, moisture, fluid conductivity, fluid chemistry, fluid composition, or location of the launch assembly.

9. The device of claim 8, wherein the detection circuitry is further structured to transmit the determined at least one characteristic to a communication device in proximity to the subject.

10. A system for delivering a therapeutic preparation to internal tissue of a subject, comprising:
a delivery device;
a carrier disposed in the delivery device, the carrier comprising the therapeutic preparation;
a launch assembly disposed in the delivery device, the launch assembly structured to cause the carrier to exit the delivery device so as to penetrate into internal tissue of the subject; and
detection circuitry structured to detect a status of deployment of the carrier.

11. The system of claim 10, further comprising a transmitter structured to transmit a signal including information on the deployment status of the carrier to a communication device external to the subject.

12. The system of claim 11, further comprising the communication device, wherein the communication device is wearable by the subject.

13. The system of claim 11, wherein the communication device is, or is incorporated into or with, a smart watch.

14. A method for delivering a therapeutic preparation into internal tissue of a subject, the method comprising:
introducing a launch assembly within the subject, the launch assembly coupled to a carrier that comprises the therapeutic preparation;
deploying, by the launch assembly, the carrier; and
detecting a status of the deployment of the carrier.

15. The method of claim 14, wherein the detection is performed by detection circuitry associated with the launch assembly.

16. The method of claim 14, wherein the detection is performed by detection circuitry associated with the carrier.

17. The method of claim 14, further comprising determining at least one of the following characteristics: pressure, velocity, acceleration, orientation, temperature, pH, torsion on a wall of the launch assembly, tension on a penetrable portion of the launch assembly, vibration, gas flow, moisture, fluid conductivity, fluid chemistry, fluid composition, or location of the launch assembly.

18. The method of claim 17, further comprising transmitting the determined at least one characteristic to an external communication device.

19. The method of claim 14, further comprising: detecting one or more conditions, and initiating the deployment of the carrier responsive to detecting the one or more conditions.

20. The method of claim 14, wherein the launch assembly is structured to deploy the carrier into or through: a wall of the stomach, a wall of the small intestine, a wall of the large intestine, or the peritoneal membrane.
